SEQUENCE LISTING (14 sequences)

SEQ ID NO: 1 - protein, 338 aa, Erwinia chrysanthemi
MQITIKAHIGGDLGVSGLGAQGLKGLNSAASSLGSSVDKLSSTIDKLTSALTSMMFGGALAQGLGASSKGLGMSNQLGQSFGNGAQGASNLLSVPKSGGDALSKMFDKALDDLLGHDTVTKLTNQSNQLANSMLNASQMTQGNMNAFGSGVNNALSSILGNGLGQSMSGFSQPSLGAGGLQGLSGAGAFNQLGNAIGMGVGQNAALSALSNVSTHVDGNNRHFVDKEDRGMAKEIGQFMDQYPEIFGKPEYQKDGWSSPKTDDKSWAKALSKPDDDGMTGASMDKFRQAMGMIKSAVAGDTGNTNLNLRGAGGASLGIDAAVVGDKIANMSLGKLANA

SEQ ID NO: 2 - DNA, 2141 bp, Erwinia chrysanthemi
cgattttacccgggtgaacgtgctatgaccgacagcatcacggtattcgacaccgttacggcgtttatggccgcgatgaaccggcatcaggcggcgcgctggtcgccgcaatccggcgtcgatctggtatttcagtttggggacaccgggcgtgaactcatgatgcagattcagccggggcagcaatatcccggcatgttgcgcacgctgctcgctcgtcgttatcagcaggcggcagagtgcgatggctgccatctgtgcctgaacggcagcgatgtattgatcctctggtggccgctgccgtcggatcccggcagttatccgcaggtgatcgaacgtttgtttgaactggcgggaatgacgttgccgtcgctatccatagcaccgacggcgcgtccgcagacagggaacggacgcgcccgatcattaagataaaggcggctttttttattgcaaaacggtaacggtgaggaaccgtttcaccgtcggcgtcactcagtaacaagtatccatcatgatgcctacatcgggatcggcgtgggcatccgttgcagatacttttgcgaacacctgacatgaatgaggaaacgaaattatgcaaattacgatcaaagcgcacatcggcggtgatttgggcgtctccggtctggggctgggtgctcagggactgaaaggactgaattccgcggcttcatcgctgggttccagcgtggataaactgagcagcaccatcgataagttgacctccgcgctgacttcgatgatgtttggcggcgcgctggcgcaggggctgggcgccagctcgaaggggctggggatgagcaatcaactgggccagtctttcggcaatggcgcgcagggtgcgagcaacctgctatccgtaccgaaatccggcggcgatgcgttgtcaaaaatgtttgataaagcgctggacgatctgctgggtcatgacaccgtgaccaagctgactaaccagagcaaccaactggctaattcaatgctgaacgccagccagatgacccagggtaatatgaatgcgttcggcagcggtgtgaacaacgcactgtcgtccattctcggcaacggtctcggccagtcgatgagtggcttctctcagccttctctgggggcaggcggcttgcagggcctgagcggcgcgggtgcattcaaccagttgggtaatgccatcggcatgggcgtggggcagaatgctgcgctgagtgcgttgagtaacgtcagcacccacgtagacggtaacaaccgccactttgtagataaagaagatcgcggcatggcgaaagagatcggccagtttatggatcagtatccggaaatattcggtaaaccggaataccagaaagatggctggagttcgccgaagacggacgacaaatcctgggctaaagcgctgagtaaaccggatgatgacggtatgaccggcgccagcatggacaaattccgtcaggcgatgggtatgatcaaaagcgcggtggcgggtgataccggcaataccaacctgaacctgcgtggcgcgggcggtgcatcgctgggtatcgatgcggctgtcgtcggcgataaaatagccaacatgtcgctgggtaagctggccaacgcctgataatctgtgctggcctgataaagcggaaacgaaaaaagagacggggaagcctgtctcttttcttattatgcggtttatgcggttacctggaccggttaatcatcgtcatcgatctggtacaaacgcacattttcccgttcattcgcgtcgttacgcgccacaatcgcgatggcatcttcctcgtcgctcagattgcgcggctgatggggaacgccgggtggaatatagagaaactcgccggccagatggagacacgtctgcgataaatctgtgccgtaacgtgtttctatccgcccctttagcagatagattgcggtttcgtaatcaacatggtaatgcggttccgcctgtgcgccggccgggatcaccacaatattcatagaaagctgtcttgcacctaccgtatcgcgggagataccgacaaaatagggcagtttttgcgtggtatccgtggggtgttccggcctgacaatcttgagttggttcgtcatcatctttctccatctgggcgacctgatcggtt

SEQ ID NO: 3 - protein, 403 aa, Erwinia amylovora
MSLNTSGLGASTMQISIGGAGGNNGLLGTSRQNAGLGGNSALGLGGGNQNDTVNQLAGLLTGMMMMMSMMGGGGLMGGGLGGGLGNGLGGSGGLGEGLSNALNDMLGGSLNTLGSKGGNNTTSTTNSPLDQALGINSTSQNDDSTSGTDSTSDSSDPMQQLLKMFSEIMQSLFGDGQDGTQGSSSGGKQPTEGEQNAYKKGVTDALSGLMGNGLSQLLGNGGLGGGQGGNAGTGLDGSSLGGKGLQNLSGPVDYQQLGNAVGTGIGMKAGIQALNDIGTHRHSSTRSFVNKGDRAMAKEIGQFMDQYPEVFGKPQYQKGPGQEVKTDDKSWAKALSKPDDDGMTPASMEQFNKAKGMIKRPMAGDTGNGNLQARGAGGSSLGIDAMMAGDAINNMALGKLGAA

SEQ ID NO: 4 - DNA, 1288 bp, Erwinia amylovora
aagcttcggcatggcacgtttgaccgttgggtcggcagggtacgtttgaattattcataagaggaatacgttatgagtctgaatacaagtgggctgggagcgtcaacgatgcaaatttctatcggcggtgcgggcggaaataacgggttgctgggtaccagtcgccagaatgctgggttgggtggcaattctgcactggggctgggcggcggtaatcaaaatgataccgtcaatcagctggctggcttactcaccggcatgatgatgatgatgagcatgatgggcggtggtgggctgatgggcggtggcttaggcggtggcttaggtaatggcttgggtggctcaggtggcctgggcgaaggactgtcgaacgcgctgaacgatatgttaggcggttcgctgaacacgctgggctcgaaaggcggcaacaataccacttcaacaacaaattccccgctggaccaggcgctgggtattaactcaacgtcccaaaacgacgattccacctccggcacagattccacctcagactccagcgacccgatgcagcagctgctgaagatgttcagcgagataatgcaaagcctgtttggtgatgggcaagatggcacccagggcagttcctctgggggcaagcagccgaccgaaggcgagcagaacgcctataaaaaaggagtcactgatgcgctgtcgggcctgatgggtaatggtctgagccagctccttggcaacgggggactgggaggtggtcagggcggtaatgctggcacgggtcttgacggttcgtcgctgggcggcaaagggctgcaaaacctgagcgggccggtggactaccagcagttaggtaacgccgtgggtaccggtatcggtatgaaagcgggcattcaggcgctgaatgatatcggtacgcacaggcacagttcaacccgttctttcgtcaataaaggcgatcgggcgatggcgaaggaaatcggtcagttcatggaccagtatcctgaggtgtttggcaagccgcagtaccagaaaggcccgggtcaggaggtgaaaaccgatgacaaatcatgggcaaaagcactgagcaagccagatgacgacggaatgacaccagccagtatggagcagttcaacaaagccaagggcatgatcaaaaggcccatggcgggtgataccggcaacggcaacctgcaggcacgcggtgccggtggttcttcgctgggtattgatgccatgatggccggtgatgccattaacaatatggcacttggcaagctgggcgcggcttaagctt

SEQ ID NO: 5 - protein, 447 aa, Erwinia amylovora
MSILTLNNNTSSSPGLFQSGGDNGLGGHNANSALGQQPIDRQTIEQMAQLLAELLKSLLSPQSGNAATGAGGNDQTTGVGNAGGLNGRKGTAGTTPQSDSQNMLSEMGNNGLDQAITPDGQGGGQIGDNPLLKAMLKLIARMMDGQSDQFGQPGTGNNSASSGTSSSGGSPFNDLSGGKAPSGNSPSGNYSPVSTFSPPSTPTSPTSPLDFPSSPTKAAGGSTPVTDHPDPVGSAGIGAGNSVAFTSAGANQTVLHDTITVKAGQVFDGKGQTFTAGSELGDGGQSENQKPLFILEDGASLKNVTMGDDGADGIHLYGDAKIDNLHVTNVGEDAITVKPNSAGKKSHVEITNSSFEHASDKILQLNADTNLSVDNVKAKDFGTFVRTNGGQQGNWDLNLSHISAEDGKFSFVKSDSEGLNVNTSDISLGDVENHYKVPMSANLKVAE

SEQ ID NO: 6 - DNA, 1344 bp, Erwinia amylovora
atgtcaattcttacgcttaacaacaatacctcgtcctcgccgggtctgttccagtccgggggggacaacgggcttggtggtcataatgcaaattctgcgttggggcaacaacccatcgatcggcaaaccattgagcaaatggctcaattattggcggaactgttaaagtcactgctatcgccacaatcaggtaatgcggcaaccggagccggtggcaatgaccagactacaggagttggtaacgctggcggcctgaacggacgaaaaggcacagcaggaaccactccgcagtctgacagtcagaacatgctgagtgagatgggcaacaacgggctggatcaggccatcacgcccgatggccagggcggcgggcagatcggcgataatcctttactgaaagccatgctgaagcttattgcacgcatgatggacggccaaagcgatcagtttggccaacctggtacgggcaacaacagtgcctcttccggtacttcttcatctggcggttccccttttaacgatctatcaggggggaaggccccttccggcaactccccttccggcaactactctcccgtcagtaccttctcacccccatccacgccaacgtcccctacctcaccgcttgatttcccttcttctcccaccaaagcagccgggggcagcacgccggtaaccgatcatcctgaccctgttggtagcgcgggcatcggggccggaaattcggtggccttcaccagcgccggcgctaatcagacggtgctgcatgacaccattaccgtgaaagcgggtcaggtgtttgatggcaaaggacaaaccttcaccgccggttcagaattaggcgatggcggccagtctgaaaaccagaaaccgctgtttatactggaagacggtgccagcctgaaaaacgtcaccatgggcgacgacggggcggatggtattcatctttacggtgatgccaaaatagacaatctgcacgtcaccaacgtgggtgaggacgcgattaccgttaagccaaacagcgcgggcaaaaaatcccacgttgaaatcactaacagttccttcgagcacgcctctgacaagatcctgcagctgaatgccgatactaacctgagcgttgacaacgtgaaggccaaagactttggtacttttgtacgcactaacggcggtcaacagggtaactgggatctgaatctgagccatatcagcgcagaagacggtaagttctcgttcgttaaaagcgatagcgaggggctaaacgtcaataccagtgatatctcactgggtgatgttgaaaaccactacaaagtgccgatgtccgccaacctgaaggtggctgaatga

SEQ ID NO: 7 - protein, 341 aa, Pseudomonas syringae
MQSLSLNSSSLQTPAMALVLVRPEAETTGSTSSKALQEVVVKLAEELMRNGQLDDSSPLGKLLAKSMAADGKAGGGIEDVIAALDKLIHEKLGDNFGASADSASGTGQQDLMTQVLNGLAKSMLDDLLTKQDGGTSFSEDDMPMLNKIAQFMDDNPAQFPKPDSGSWVNELKEDNFLDGDETAAFRSALDIIGQQLGNQQSDAGSLAGTGGGLGTPSSFSNNSSVMGDPLIDANTGPGDSGNTRGEAGQLIGELIDRGLQSVLAGGGLGTPVNTPQTGTSANGGQSAQDLDQLLGGLLLKGLEATLKDAGQTGTDVQSSAAQIATLLVSTLLQGTRNQAAA

SEQ ID NO: 8 - DNA, 1026 bp, Pseudomonas syringae
atgcagagtctcagtcttaacagcagctcgctgcaaaccccggcaatggcccttgtcctggtacgtcctgaagccgagacgactggcagtacgtcgagcaaggcgcttcaggaagttgtcgtgaagctggccgaggaactgatgcgcaatggtcaactcgacgacagctcgccattgggaaaactgttggccaagtcgatggccgcagatggcaaggcgggcggcggtattgaggatgtcatcgctgcgctggacaagctgatccatgaaaagctcggtgacaacttcggcgcgtctgcggacagcgcctcgggtaccggacagcaggacctgatgactcaggtgctcaatggcctggccaagtcgatgctcgatgatcttctgaccaagcaggatggcgggacaagcttctccgaagacgatatgccgatgctgaacaagatcgcgcagttcatggatgacaatcccgcacagtttcccaagccggactcgggctcctgggtgaacgaactcaaggaagacaacttccttgatggcgacgaaacggctgcgttccgttcggcactcgacatcattggccagcaactgggtaatcagcagagtgacgctggcagtctggcagggacgggtggaggtctgggcactccgagcagtttttccaacaactcgtccgtgatgggtgatccgctgatcgacgccaataccggtcccggtgacagcggcaatacccgtggtgaagcggggcaactgatcggcgagcttatcgaccgtggcctgcaatcggtattggccggtggtggactgggcacacccgtaaacaccccgcagaccggtacgtcggcgaatggcggacagtccgctcaggatcttgatcagttgctgggcggcttgctgctcaagggcctggaggcaacgctcaaggatgccgggcaaacaggcaccgacgtgcagtcgagcgctgcgcaaatcgccaccttgctggtcagtacgctgctgcaaggcacccgcaatcaggctgcagcctga

SEQ ID NO: 9 - protein, 424 aa, Pseudomonas syringae
MSIGITPRPQQTTTPLDFSALSGKSPQPNTFGEQNTQQAIDPSALLFGSDTQKDVNFGTPDSTVQNPQDASKPNDSQSNIAKLISALIMSLLQMLTNSNKKQDTNQEQPDSQAPFQNNGGLGTPSADSGGGGTPDATGGGGGDTPSATGGGGGDTPTATGGGGSGGGGTPTATGGGSGGTPTATGGGEGGVTPQITPQLANPNRTSGTGSVSDTAGSTEQAGKINVVKDTIKVGAGEVFDGHGATFTADKSMGNGDQGENQKPMFELAEGATLKNVNLGENEVDGIHVKAKNAQEVTIDNVHAQNVGEDLITVKGEGGAAVTNLNIKNSSAKGADDKVVQLNANTHLKIDNFKADDFGTMVRTNGGKQFDDMSIELNGIEANHGKFALVKSDSDDLKLATGNIAMTDVKHAYDKTQASTQHTEL

SEQ ID NO: 10 - DNA, 1729 bp, Pseudomonas syringae
tccacttcgctgattttgaaattggcagattcatagaaacgttcaggtgtggaaatcaggctgagtgcgcagatttcgttgataagggtgtggtactggtcattgttggtcatttcaaggcctctgagtgcggtgcggagcaataccagtcttcctgctggcgtgtgcacactgagtcgcaggcataggcatttcagttccttgcgttggttgggcatataaaaaaaggaacttttaaaaacagtgcaatgagatgccggcaaaacgggaaccggtcgctgcgctttgccactcacttcgagcaagctcaaccccaaacatccacatccctatcgaacggacagcgatacggccacttgctctggtaaaccctggagctggcgtcggtccaattgcccacttagcgaggtaacgcagcatgagcatcggcatcacaccccggccgcaacagaccaccacgccactcgatttttcggcgctaagcggcaagagtcctcaaccaaacacgttcggcgagcagaacactcagcaagcgatcgacccgagtgcactgttgttcggcagcgacacacagaaagacgtcaacttcggcacgcccgacagcaccgtccagaatccgcaggacgccagcaagcccaacgacagccagtccaacatcgctaaattgatcagtgcattgatcatgtcgttgctgcagatgctcaccaactccaataaaaagcaggacaccaatcaggaacagcctgatagccaggctcctttccagaacaacggcgggctcggtacaccgtcggccgatagcgggggcggcggtacaccggatgcgacaggtggcggcggcggtgatacgccaagcgcaacaggcggtggcggcggtgatactccgaccgcaacaggcggtggcggcagcggtggcggcggcacacccactgcaacaggtggcggcagcggtggcacacccactgcaacaggcggtggcgagggtggcgtaacaccgcaaatcactccgcagttggccaaccctaaccgtacctcaggtactggctcggtgtcggacaccgcaggttctaccgagcaagccggcaagatcaatgtggtgaaagacaccatcaaggtcggcgctggcgaagtctttgacggccacggcgcaaccttcactgccgacaaatctatgggtaacggagaccagggcgaaaatcagaagcccatgttcgagctggctgaaggcgctacgttgaagaatgtgaacctgggtgagaacgaggtcgatggcatccacgtgaaagccaaaaacgctcaggaagtcaccattgacaacgtgcatgcccagaacgtcggtgaagacctgattacggtcaaaggcgagggaggcgcagcggtcactaatctgaacatcaagaacagcagtgccaaaggtgcagacgacaaggttgtccagctcaacgccaacactcacttgaaaatcgacaacttcaaggccgacgatttcggcacgatggttcgcaccaacggtggcaagcagtttgatgacatgagcatcgagctgaacggcatcgaagctaaccacggcaagttcgccctggtgaaaagcgacagtgacgatctgaagctggcaacgggcaacatcgccatgaccgacgtcaaacacgcctacgataaaacccaggcatcgacccaacacaccgagctttgaatccagacaagtagcttgaaaaaagggggtggactc

SEQ ID NO: 11 - protein, 344 aa, Ralstonia solanacearum
MSVGNIQSPSNLPGLQNLNLNTNTNSQQSGQSVQDLIKQVEKDILNIIAALVQKAAQSAGGNTGNTGNAPAKDGNANAGANDPSKNDPSKSQAPQSANKTGNVDDANNQDPMQALMQLLEDLVKLLKAALHMQQPGGNDKGNGVGGANGAKGAGGQGGLAEALQEIEQILAQLGGGGAGAGGAGGGVGGAGGADGGSGAGGAGGANGADGGNGVNGNQANGPQNAGDVNGANGADDGSEDQGGLTGVLQKLMKILNALVQMMQQGGLGGGNQAQGGSKGAGNASPASGANPGANQPGSADDQSSGQNNLQSQIMDVVKEVVQILQQMLAAQNGGSQQSTSTQPM

SEQ ID NO: 12 - DNA, 1035 bp, Ralstonia solanacearum
atgtcagtcggaaacatccagagcccgtcgaacctcccgggtctgcagaacctgaacctcaacaccaacaccaacagccagcaatcgggccagtccgtgcaagacctgatcaagcaggtcgagaaggacatcctcaacatcatcgcagccctcgtgcagaaggccgcacagtcggcgggcggcaacaccggtaacaccggcaacgcgccggcgaaggacggcaatgccaacgcgggcgccaacgacccgagcaagaacgacccgagcaagagccaggctccgcagtcggccaacaagaccggcaacgtcgacgacgccaacaaccaggatccgatgcaagcgctgatgcagctgctggaagacctggtgaagctgctgaaggcggccctgcacatgcagcagcccggcggcaatgacaagggcaacggcgtgggcggtgccaacggcgccaagggtgccggcggccagggcggcctggccgaagcgctgcaggagatcgagcagatcctcgcccagctcggcggcggcggtgctggcgccggcggcgcgggtggcggtgtcggcggtgctggtggcgcggatggcggctccggtgcgggtggcgcaggcggtgcgaacggcgccgacggcggcaatggcgtgaacggcaaccaggcgaacggcccgcagaacgcaggcgatgtcaacggtgccaacggcgcggatgacggcagcgaagaccagggcggcctcaccggcgtgctgcaaaagctgatgaagatcctgaacgcgctggtgcagatgatgcagcaaggcggcctcggcggcggcaaccaggcgcagggcggctcgaagggtgccggcaacgcctcgccggcttccggcgcgaacccgggcgcgaaccagcccggttcggcggatgatcaatcgtccggccagaacaatctgcaatcccagatcatggatgtggtgaaggaggtcgtccagatcctgcagcagatgctggcggcgcagaacggcggcagccagcagtccacctcgacgcagccgatgtaa

SEQ ID NO: 13 - protein, 114 aa, Xanthomonas campestris
MDSIGNNFSNIGNLQTMGIGPQQHEDSSQQSPSAGSEQQLDQLLAMFIMMMLQQSQGSDANQECGNEQPQNGQQEGLSPLTQMLMQIVMQLMQNQGGAGMGGGGSVNSSLGGNA

SEQ ID NO: 14 - DNA, 342 bp, Xanthomonas campestris
atggactctatcggaaacaacttttcgaatatcggcaacctgcagacgatgggcatcgggcctcagcaacacgaggactccagccagcagtcgccttcggctggctccgagcagcagctggatcagttgctcgccatgttcatcatgatgatgctgcaacagagccagggcagcgatgcaaatcaggagtgtggcaacgaacaaccgcagaacggtcaacaggaaggcctgagtccgttgacgcagatgctgatgcagatcgtgatgcagctgatgcagaaccagggcggcgccggcatgggcggtggcggttcggtcaacagcagcctgggcggcaacgcc

(19) United States
(12) Patent Application Publication
(10) Pub. No.: US 2003/0104979 A1
(43) Pub. Date: Jun. 5, 2003

(54) Methods of inhibiting desiccation of cuttings removed from ornamental plants

(76) Inventors: Zhong-Min Wei, Kirkland, WA (US); Ernesto Leon, Coyacan (MX); Agustin Oviedo, Celaya (MX)

Correspondence Address: Michael L. Goldman, NIXON PEABODY LLP, Clinton Square, P.O. Box 31051, Rochester, NY, 14603, US

(21) Appl. No.: 10/010,390

(22) Filed: Nov. 5, 2001

Related U.S. Application Data
(60) Provisional application No. 60/248,169, filed on Nov. 13, 2000.

(51) Int. Cl. A01N 37/18; A61K 38/00; A01H 5/00

(52) U.S. Cl. 514/2; 800/323

(57) ABSTRACT

Disclosed are methods of inhibiting desiccation of cuttings from ornamental plants, methods of harvesting cuttings from ornamental plants, methods of promoting early flowering of ornamental plants, and methods of enhancing the longevity of flower blooms on ornamental plant cuttings. The ornamental plants can be transgenic plants which express a heterologous hypersensitive response elicitor protein or polypeptide or the ornamental plants can be treated via topical application with a hypersensitive response elicitor protein or polypeptide. Alternatively, cuttings from the ornamental plant can be treated with a hypersensitive response elicitor protein or polypeptide, independent of any treatment provided to the ornamental plant from which the cutting is removed.

This application claims benefit of U.S. Provisional Patent Application Serial No. 60/248,169, filed Nov. 13, 2000, which is hereby incorporated by reference in its entirety.

### FIELD OF THE INVENTION

The present invention generally relates to methods of treating ornamental plants or cuttings removed therefrom to inhibit desiccation of cuttings removed from the ornamental plants.

### BACKGROUND OF THE INVENTION

According to an April 2001 report by the United States Department of Agriculture, National Agricultural Statistics Service, Sp Cr 6-1 (01), entitled “Floriculture Crops: 2000 Summary”, during the previous year the wholesale value of domestically produced cut flowers was $427 million. The top three valued cut flower categories were Roses at $69.4 million, Lilies at $58.6 million, and Gladioli at $32.2 million. While the U.S. cut flower industry is not insignificant, two-thirds of the cut flowers sold in the U.S. in 1998 were imported, and this import market was worth $1 billion. Of the imports coming into the U.S. that year, 56% were from Colombia, 22% from elsewhere in Central & South America, and about 18% from The Netherlands.

Postharvest handling methods that were developed over 20 years ago on U.S. produced flowers are still current practice in the fresh flower industry. However, as noted above, many flowers sold in the U.S. today are imported from Colombia and Ecuador and can be 8-10 days old when purchased by consumers. Current problems with cut flower longevity and quality are associated with shifts in the geographical locations of production, introduction of new varieties, long-distance transport from farm to consumer, improper transport and storage temperatures, and undesirable handling practices. With respect to transport and storage temperatures, prevalent problems include: flowers are often not pre-cooled adequately when they leave the grower; use of non-refrigerated trucks during shipment; boxed flowers which sit for extended periods on non-refrigerated docks; and flowers are not kept cool during air transport.

The effect that these problems can have on cut flower longevity includes not only poor appearance of flowers at retail sites, but also loss of flowers (i.e., wilting or dying) prior to the time they reach the retailer or shortly thereafter. In either case, the wholesaler or the retailer may realize financial losses as a result.

A number of strategies have been devised to minimize flower loss. These include treatment with silver thiosulfate, 1-methylcyclopropene (MCP), carboxymethoxylamine (also known as aminooxyacetic acid (AOAA)), AVG, N-AVG, rhizobitoxine, or L-trans-2-amino-4-methoxy-3-butenoic acid (MVG). Silver thiosulfate and MCP are believed to inhibit the effect of either internal or external ethylene, while the others are believed to act internally to inhibit the ability of the cut flowers, plants, and fruit to produce ethylene. These compounds (except MCP) are typically applied to plants or plant materials in the form of an aqueous treatment solution. Applications of the treatment solution to potted plants are carried out by spraying it onto the aerial parts of the plants or by including it in the irrigation water which is supplied to their roots. Treatment of cut flowers or greens is typically carried out by immersing the cut ends of the stems in the aqueous solution containing the treating agent immediately after harvest, during transportation or while the floral arrangement is on display, although they might be treated by immersing the whole flowers into a solution or by spraying them. Since MCP is a gas, it cannot readily be applied in aqueous solution, so plants are treated by exposing them to a modified, controlled atmosphere (containing a defined amount of MCP) in an enclosed chamber.

Silver thiosulfate is expensive and it may be toxic to animals. Although MCP is now commercially available, its use is limited due to difficulties in application and its lack of stability.

However effective these earlier attempts to reduce cut flower losses, there still exists a need to provide improved, non-toxic and easily practiced approaches for minimizing the losses of ornamental plant cuttings. The present invention is directed to overcoming these deficiencies in the art.

### SUMMARY OF THE INVENTION

A first aspect of the present invention relates to a method of inhibiting desiccation of cuttings from ornamental plants which includes: treating an ornamental plant with a hypersensitive response elicitor protein or polypeptide under conditions effective to inhibit desiccation of a cutting from the ornamental plant after the cutting is removed from the ornamental plant.

A second aspect of the present invention relates to a cutting which has been removed from an ornamental plant treated with a hypersensitive response elicitor protein or polypeptide, wherein the cutting is characterized by greater resistance to desiccation as compared to a cutting removed from an untreated ornamental plant.

A third aspect of the present invention relates to a method of promoting early flowering of an ornamental plant which includes: treating an ornamental plant with a hypersensitive response elicitor protein or polypeptide under conditions effective to promote early flowering of the ornamental plant.

A fourth aspect of the present invention relates to a method of harvesting a cutting from an ornamental plant which includes: treating an ornamental plant with a hypersensitive response elicitor protein or polypeptide and harvesting a cutting from the treated ornamental plant.

A fifth aspect of the present invention relates to a method of harvesting a cutting from an ornamental plant which includes: harvesting a cutting from an ornamental plant and treating the harvested cutting with a hypersensitive response elicitor protein or polypeptide.

A sixth aspect of the present invention relates to a method of inhibiting desiccation of cuttings from ornamental plants which includes: removing a cutting from an ornamental plant and treating the removed cutting with a hypersensitive response elicitor protein or polypeptide under conditions effective to inhibit desiccation of the removed cutting.

A seventh aspect of the present invention relates to a cutting which has been removed from an ornamental plant, wherein the cutting has been treated with a hypersensitive response elicitor protein or polypeptide and wherein the cutting is characterized by greater resistance to desiccation as compared to an untreated cutting removed from the ornamental plant.

An eight aspect of the present invention relates to a method of inhibiting desiccation of cuttings from ornamental plants which includes: providing a transgenic ornamental plant or plant seed transformed with a DNA molecule encoding a hypersensitive response elicitor polypeptide or protein and growing the transgenic ornamental plant or transgenic ornamental plant produced from the transgenic ornamental plant seed under conditions effective to inhibit desiccation in a cutting removed from the transgenic plant.

A ninth aspect of the present invention relates to a method of promoting early flowering of an ornamental plant which includes: providing a transgenic ornamental plant or plant seed transformed with a DNA molecule encoding a hypersensitive response elicitor polypeptide or protein and growing the transgenic ornamental plant or transgenic ornamental plant produced from the transgenic ornamental plant seed under conditions effective to promote early flowering of the transgenic ornamental plant.

A tenth aspect of the present invention relates to a method of harvesting a cutting from an ornamental plant which includes: providing a transgenic ornamental plant or plant seed transformed with a DNA molecule encoding a hypersensitive response elicitor polypeptide or protein; growing the transgenic ornamental plant or transgenic ornamental plant produced from the transgenic ornamental plant seed under conditions; and harvesting a cutting from the grown transgenic ornamental plant, wherein the cutting exhibits a reduced susceptibility to desiccation as compared to cuttings removed from non-transgenic ornamental plants.

An eleventh aspect of the present invention relates to a cutting which has been removed from a transgenic ornamental plant which expresses a heterologous hypersensitive response elicitor protein or polypeptide, wherein the cutting is characterized by greater resistance to desiccation as compared to a cutting removed from a non-transgenic ornamental plant.

A twelfth aspect of the present invention relates to a method of enhancing the longevity of flower blooms on ornamental plant cuttings which includes: providing a transgenic ornamental plant or plant seed transformed with a DNA molecule encoding a hypersensitive response elicitor polypeptide or protein and growing the transgenic ornamental plant or transgenic ornamental plant produced from the transgenic ornamental plant seed under conditions effective to enhancing the longevity of flower blooms on cuttings removed therefrom.

A thirteenth aspect of the present invention relates to a method of enhancing the longevity of flower blooms on ornamental plant cuttings which includes: treating an ornamental plant with a hypersensitive response elicitor protein or polypeptide under conditions effective to enhancing the longevity of flower blooms on cuttings removed therefrom.

A fourteenth aspect of the present invention relates to a method of enhancing the longevity of flower blooms on ornamental plant cuttings which includes: harvesting a cutting from an ornamental plant and treating the harvested cutting with a hypersensitive response elicitor protein or polypeptide under conditions effective to enhancing the longevity of flower blooms on the harvested cutting.

Because hypersensitive response elicitor proteins or polypeptides can easily be expressed transgenically in or applied topically to ornamental plants and/or ornamental plant cuttings, the present invention offers an effective, simple-to-use, non-toxic approach for inhibiting the desiccation of cuttings removed from ornamental plants, promoting early flowering of the ornamental plants, and enhancing the longevity of flower blooms on ornamental plant cuttings. By inhibiting desiccation of cuttings after they have been removed from an ornamental plant, the cuttings are less likely to wilt and die before they are received by the retailer. This will dramatically decrease losses associated with long transportation rates in less than ideal conditions. Moreover, it is also possible to enhancing the longevity of flower blooms, which end consumers can clearly appreciate.

### BRIEF DESCRIPTION OF THE DRAWINGS

FIG. 1 is an image illustrating the response of Vega roses to pre- and postharvest application of EBC-151 (left), untreated (center), and preharvest only treatment with EBC-151. Image captured 16 days after harvest and postharvest treatment with EBC-151.

FIG. 2 is an image illustrating the response of Vega roses to pre-harvest only applications of EBC-151; 150+350 g/Ha (left), untreated (center), and 250 g/Ha (right). Image captured 16 days after harvest; no postharvest treatment applied.

FIG. 3 is an image illustrating the response of Vega roses to postharvest only application of EBC-151. Image captured 16 days after harvest.

### DETAILED DESCRIPTION OF THE INVENTION

The present invention relates to methods of inhibiting desiccation of cuttings from ornamental plants, methods of harvesting cuttings from ornamental plants, methods of promoting early flowering of ornamental plants, and methods of enhancing the longevity of flower blooms on ornamental plant cuttings.

The ornamental plants can be transgenic plants which express a heterologous hypersensitive response elicitor protein or polypeptide or the ornamental plants can be treated (i.e., via topical application) with a hypersensitive response elicitor protein or polypeptide. Alternatively, the cutting from the ornamental plant (whether transgenic or not) can itself be treated with a hypersensitive response elicitor protein or polypeptide, independent of any treatment provided to the ornamental plant from which the cutting is removed.

For use in accordance with these methods, suitable hypersensitive response elicitor proteins or polypeptides are those derived from a wide variety of bacterial and fungal pathogens, preferably bacterial pathogens.

Exemplary hypersensitive response elicitor proteins and polypeptides from bacterial sources include, without limitation, the hypersensitive response elicitors derived from Erwinia species (e.g., Erwinia amylovora, Erwinia chrysanthemi, Erwinia stewartii, Erwinia carotovora, etc.), Pseudomonas species (e.g., Pseudomonas syringae), Ralstonia species (e.g., Ralstonia solanacearum), and Xanthomonas species (e.g., Xanthomonas campestris). In addition to hypersensitive response elicitors from these Gram-negative bacteria, it is possible to use elicitors derived from Gram-positive bacteria. One example is the hypersensitive response elicitor derived from Clavibacter michiganensis subsp. sepedonicus.

Exemplary hypersensitive response elicitor proteins or polypeptides from fungal sources include, without limitation, the hypersensitive response elicitors (i.e., elicitins) from various Phytophthora species (e.g., Phytophthora parasitica, Phytophthora cryptogea, Phytophthora cinnamomi, Phytophthora capsici, Phytophthora megasperma, Phytophthora citrophthora, etc.).

Preferably, the hypersensitive response elicitor protein or polypeptide is derived from Erwinia chrysanthemi, Erwinia amylovora, Pseudomonas syringae, Ralstonia solanacearum, or Xanthomonas campestris.

A hypersensitive response elicitor protein or polypeptide from Erwinia chrysanthemi has an amino acid sequence corresponding to SEQ. ID. No. 1 as follows:

```
Met Gln Ile Thr Ile Lys Ala His Ile Gly Gly Asp
1 5 10
Leu Gly Val Ser Gly Leu Gly Ala Gln Gly Leu Lys
                15 20
Gly Leu Asn Ser Ala Ala Ser Ser Leu Gly Ser Ser
25 30 35
Val Asp Lys Leu Ser Ser Thr Ile Asp Lys Leu Thr
                        40 45
Ser Ala Leu Thr Ser Met Met Phe Gly Gly Ala Leu
        50 55 60
Ala Gln Gly Leu Gly Ala Ser Ser Lys Gly Leu Gly
65 70
Met Ser Asn Gln Leu Gly Gln Ser Phe Gly Asn Gly
                75 80
Ala Gln Gly Ala Ser Asn Leu Leu Ser Val Pro Lys
85 90 95
Ser Gly Gly Asp Ala Leu Ser Lys Met Phe Asp Lys
                        100 105
Ala Leu Asp Asp Leu Leu Gly His Asp Thr Val Thr
        110 115 120
Lys Leu Thr Asn Gln Ser Asn Gln Leu Ala Asn Ser
                                                  125 130
Met Leu Asn Ala Ser Gln Met Thr Gln Gly Asn Met
                135 140
Asn Ala Phe Gly Ser Gly Val Asn Asn Ala Leu Ser
145 150 155
Ser Ile Leu Gly Asn Gly Leu Gly Gln Ser Met Ser
                        160 165
Gly Phe Ser Gln Pro Ser Leu Gly Ala Gly Gly Leu
        170 175 180
Gln Gly Leu Ser Gly Ala Gly Ala Phe Asn Gln Leu
                                185 190
Gly Asn Ala Ile Gly Met Gly Val Gly Gln Asn Ala
                195 200
Ala Leu Ser Ala Leu Ser Asn Val Ser Thr His Val
205 210 215
Asp Gly Asn Asn Arg His Phe Val Asp Lys Glu Asp
                220 225
Arg Gly Met Ala Lys Glu Ile Gly Gln Phe Met Asp
        230 235 240
Gln Tyr Pro Glu Ile Phe Gly Lys Pro Glu Tyr Gln
                                245 250
Lys Asp Gly Trp Ser Ser Pro Lys Thr Asp Asp Lys
                255 260
Ser Trp Ala Lys Ala Leu Ser Lys Pro Asp Asp Asp
265 270 275
Gly Met Thr Gly Ala Ser Met Asp Lys Phe Arg Gln
                        280 285
Ala Met Gly Met Ile Lys Ser Ala Val Ala Gly Asp
        290 295 300
Thr Gly Asn Thr Asn Leu Asn Leu Arg Gly Ala Gly
                                305 310
Gly Ala Ser Leu Gly Ile Asp Ala Ala Val Val Gly
                315 320
Asp Lys Ile Ala Asn Met Ser Leu Gly Lys Leu Ala
325 330 335
Asn Ala
```

This hypersensitive response elicitor protein or polypeptide has a molecular mass of 34 kDa, is heat stable, has a glycine content of greater than 16%, and contains substantially no cysteine. This Erwinia chrysanthemi hypersensitive response elicitor protein or polypeptide is encoded by a DNA molecule having a nucleotide sequence corresponding to SEQ. ID. No. 2 as follows:

| | |
|---|---|
| cgattttacc cgggtgaacg tgctatgacc gacagcatca | 60 |

cggtattcga caccgttacg

| | |
|---|---|
| gcgtttatgg ccgcgatgaa ccggcatcag gcggcgcgct | 120 |

ggtcgccgca atccggcgtc

| | |
|---|---|
| gatctggtat ttcagtttgg ggacaccggg cgtgaactca | 180 |

tgatgcagat tcagccgggg

| | |
|---|---|
| cagcaatatc ccggcatgtt gcgcacgctg ctcgctcgtc | 240 |

gttatcagca ggcggcagag

| | |
|---|---|
| tgcgatggct gccatctgtg cctgaacggc agcgatgtat | 300 |

tgatcctctg gtggccgctg

| | |
|---|---|
| ccgtcggatc ccggcagtta tccgcaggtg atcgaacgtt | 360 |

tgtttgaact ggcgggaatg

| | |
|---|---|
| acgttgccgt cgctatccat agcaccgacg gcgcgtccgc | 420 |

agacagggaa cggacgcgcc

| | |
|---|---|
| cgatcattaa gataaaggcg gcttttttta ttgcaaaacg | 480 |

gtaacggtga ggaaccgttt

| | |
|---|---|
| caccgtcggc gtcactcagt aacaagtatc catcatgatg | 540 |

cctacatcgg gatcggcgtg

| | |
|---|---|
| ggcatccgtt gcagatactt ttgcgaacac ctgacatgaa | 600 |

tgaggaaacg aaattatgca

| | |
|---|---|
| aattacgatc aaagcgcaca tcggcggtga tttgggcgtc | 660 |

tccggtctgg ggctgggtgc

| | |
|---|---|
| tcagggactg aaaggactga attccgcggc ttcatcgctg | 720 |

ggttccagcg tggataaact

| | |
|---|---|
| gagcagcacc atcgataagt tgacctccgc gctgacttcg | 780 |

atgatgtttg gcggcgcgct

| | |
|---|---|
| ggcgcagggg ctgggcgcca gctcgaaggg gctggggatg | 840 |

agcaatcaac tgggccagtc

| | |
|---|---|
| tttcggcaat ggcgcgcagg gtgcgagcaa cctgctatcc | 900 |

gtaccgaaat ccggcggcga

| | |
|---|---|
| tgcgttgtca aaaatgtttg ataaagcgct ggacgatctg | 960 |

ctgggtcatg acaccgtgac

| | |
|---|---|
| caagctgact aaccagagca accaactggc taattcaatg | 1020 |

ctgaacgcca gccagatgac

| | |
|---|---|
| ccagggtaat atgaatgcgt tcggcagcgg tgtgaacaac | 1080 |

gcactgtcgt ccattctcgg

| | |
|---|---|
| caacggtctc ggccagtcga tgagtggctt ctctcagcct | 1140 |

tctctggggg caggcggctt

| | |
|---|---|
| gcagggcctg agcggcgcgg gtgcattcaa ccagttgggt | 1200 |

aatgccatcg gcatgggcgt

| | |
|---|---|
| ggggcagaat gctgcgctga gtgcgttgag taacgtcagc | 1260 |

acccacgtag acggtaacaa

| | |
|---|---|
| ccgccacttt gtagataaag aagatcgcgg catggcgaaa | 1320 |

gagatcggcc agtttatgga

| | |
|---|---|
| tcagtatccg gaaatattcg gtaaaccgga ataccagaaa | 1380 |

gatggctgga gttcgccgaa

| | |
|---|---|
| gacggacgac aaatcctggg ctaaagcgct gagtaaaccg | 1440 |

gatgatgacg gtatgaccgg

| | |
|---|---|
| cgccagcatg gacaaattcc gtcaggcgat gggtatgatc | 1500 |

aaaagcgcgg tggcgggtga

| | |
|---|---|
| taccggcaat accaacctga acctgcgtgg cgcgggcggt | 1560 |

gcatcgctgg gtatcgatgc

| | |
|---|---|
| ggctgtcgtc ggcgataaaa tagccaacat gtcgctgggt | 1620 |

aagctggcca acgcctgata

| | |
|---|---|
| atctgtgctg gcctgataaa gcggaaacga aaaaagagac | 1680 |

ggggaagcct gtctcttttc

| | |
|---|---|
| ttattatgcg gtttatgcgg ttacctggac cggttaatca | 1740 |

tcgtcatcga tctggtacaa

| | |
|---|---|
| acgcacattt tcccgttcat tcgcgtcgtt acgcgccaca | 1800 |

atcgcgatgg catcttcctc

| | |
|---|---|
| gtcgctcaga ttgcgcggct gatggggaac gccgggtgga | 1860 |

atatagagaa actcgccggc

| | |
|---|---|
| cagatggaga cacgtctgcg ataaatctgt gccgtaacgt | 1920 |

gtttctatcc gcccctttag

| | |
|---|---|
| cagatagatt gcggtttcgt aatcaacatg gtaatgcggt | 1980 |

tccgcctgtg cgccggccgg

| | |
|---|---|
| gatcaccaca atattcatag aaagctgtct tgcacctacc | 2040 |

gtatcgcggg agataccgac

| | |
|---|---|
| aaaatagggc agtttttgcg tggtatccgt ggggtgttcc | 2100 |

ggcctgacaa tcttgagttg

| | |
|---|---|
| gttcgtcatc atctttctcc atctgggcga cctgatcggt t | 2141 |

The above nucleotide and amino acid sequences are disclosed and further described in U.S. Pat. No. 5,850,015 to Bauer et al. and U.S. Pat. No. 5,776,889 to Wei et al., each of which is hereby incorporated by reference in its entirety.

A hypersensitive response elicitor protein or polypeptide derived from Erwinia amylovora has an amino acid sequence corresponding to SEQ. ID. No. 3 as follows:

```
Met Ser Leu Asn Thr Ser Gly Leu Gly Ala Ser Thr
1 5 10
Met Gln Ile Ser Ile Gly Gly Ala Gly Gly Asn Asn
                15 20
Gly Leu Leu Gly Thr Ser Arg Gln Asn Ala Gly Leu
25 30 35
Gly Gly Asn Ser Ala Leu Gly Leu Gly Gly Gly Asn
                        40 45
Gln Asn Asp Thr Val Asn Gln Leu Ala Gly Leu Leu
        50 55 60
Thr Gly Met Met Met Met Met Ser Met Met Gly Gly
                                65 70
Gly Gly Leu Met Gly Gly Gly Leu Gly Gly Gly Leu
                75 80
Gly Asn gly Leu Gly Gly Ser Gly Gly Leu Gly Glu
85 90 95
Gly Leu Ser Asn Ala Leu Asn Asp Met Leu Gly Gly
                        100 105
Ser Leu Asn Thr Leu Gly Ser Lys Gly Gly Asn Asn
        110 115 120
Thr Thr Ser Thr Thr Asn Ser Pro Leu Asp Gln Ala
                                          125 130
Leu Gly Ile Asn Ser Thr Ser Gln Asn Asp Asp Ser
                135 140
Thr Ser Gly Thr Asp Ser Thr Ser Asp Ser Ser Asp
145 150 155
Pro Met Gln Gln Leu Leu Lys Met Phe Ser Glu Ile
                        160 165
Met Gln Ser Leu Phe Gly Asp Gly Gln Asp Gly Thr
        170 175 180
Gln Gly Ser Ser Ser Gly Gly Lys Gln Pro Thr Glu
                                  185 190
Gly Glu Gln Asn Ala Tyr Lys Lys Gly Val Thr Asp
                195 200
Ala Leu Ser Gly Leu Met Gly Asn Gly Leu Ser Gln
205 210 215
Leu Leu Gly Asn Gly Gly Leu Gly Gly Gly Gln Gly
                        220 225
Gly Asn Ala Gly Thr Gly Leu Asp Gly Ser Ser Leu
          230 235 240
Gly Gly Lys Gly Leu Gln Asn Leu Ser Gly Pro Val
                                245 250
Asp Tyr Gln Gln Leu Gly Asn Ala Val Gly Thr Gly
                255 260
Ile Gly Met Lys Ala Gly Ile Gln Ala Leu Asn Asp
265 270 275
Ile Gly Thr His Arg His Ser Ser Thr Arg Ser Phe
                        280 285
Val Asn Lys Gly Asp Arg Ala Met Ala Lys Glu Ile
        290 295 300
Gly Gln Phe Met Asp Gln Tyr Pro Glu Val Phe Gly
305 310
Lys Pro Gln Tyr Gln Lys Gly Pro Gly Gln Glu Val
                315 320
Lys Thr Asp Asp Lys Ser Trp Ala Lys Ala Leu Ser
325 330 335
Lys Pro Asp Asp Asp Gly Met Thr Pro Ala Ser Met
                        340 345
Glu Gln Phe Asn Lys Ala Lys Gly Met Ile Lys Arg
        350 355 360
Pro Met Ala Gly Asp Thr Gly Asn Gly Asn Leu Gln
                                365 370
Ala Arg Gly Ala Gly Gly Ser Ser Leu Gly Ile Asp
                375 380
Ala Met Met Ala Gly Asp Ala Ile Asn Asn Met Ala
385 390 395
Leu Gly Lys Leu Gly Ala Ala
                        400
```

This hypersensitive response elicitor protein or polypeptide has a molecular mass of about 39 kDa, has a pI of approximately 4.3, and is heat stable at 100° C. for at least 10 minutes. This hypersensitive response elicitor protein or polypeptide has substantially no cysteine. The hypersensitive response elicitor protein or polypeptide derived from Erwinia amylovora is more fully described in Wei, Z-M., et al., “Harpin, Elicitor of the Hypersensitive Response Produced by the Plant Pathogen Erwinia amylovora,” Science 257:85-88 (1992), which is hereby incorporated by reference in its entirety. The DNA molecule encoding this hypersensitive response elicitor protein or polypeptide has a nucleotide sequence corresponding to SEQ. ID. No. 4 as follows:

| | |
|---|---|
| aagcttcggc atggcacgtt tgaccgttgg gtcggcaggg | 60 |

tacgtttgaa ttattcataa

| | |
|---|---|
| gaggaatacg ttatgagtct gaatacaagt gggctgggag | 120 |

cgtcaacgat gcaaatttct

| | |
|---|---|
| atcggcggtg cgggcggaaa taacgggttg ctgggtacca | 180 |

gtcgccagaa tgctgggttg

| | |
|---|---|
| ggtggcaatt ctgcactggg gctgggcggc ggtaatcaaa | 240 |

atgataccyt caatcagctg

| | |
|---|---|
| gctggcttac tcaccggcat gatgatgatg atgagcatga | 300 |

tgggcggtgg tgggctgatg

| | |
|---|---|
| ggcggtggct taggcggtgg cttaggtaat ggcttgggtg | 360 |

gctcaggtgg cctgggcgaa

| | |
|---|---|
| ggactgtcga acgcgctgaa cgatatgtta ggcggttcgc | 420 |

tgaacacgct gggctcgaaa

| | |
|---|---|
| ggcggcaaca ataccacttc aacaacaaat tccccgctgg | 480 |

accaggcgct gggtattaac

| | |
|---|---|
| tcaacgtccc aaaacgacga ttccacctcc ggcacagatt | 540 |

ccacctcaga ctccagcgac

| | |
|---|---|
| ccgatgcagc agctgctgaa gatgttcagc gagataatgc | 600 |

aaagcctgtt tggtgatggg

| | |
|---|---|
| caagatggca cccagggcag ttcctctggg ggcaagcagc | 660 |

cgaccgaagg cgagcagaac

| | |
|---|---|
| gcctataaaa aaggagtcac tgatgcgctg tcgggcctga | 720 |

tgggtaatgg tctgagccag

| | |
|---|---|
| ctccttggca acgggggact gggaggtggt cagggcggta | 780 |

atgctggcac gggtcttgac

| | |
|---|---|
| ggttcgtcgc tgggcggcaa agggctgcaa aacctgagcg | 840 |

ggccggtgga ctaccagcag

| | |
|---|---|
| ttaggtaacg ccgtgggtac cggtatcggt atgaaagcgg | 900 |

gcattcaggc gctgaatgat

| | |
|---|---|
| atcggtacgc acaggcacag ttcaacccgt tctttcgtca | 960 |

ataaaggcga tcgggcgatg

| | |
|---|---|
| gcgaaggaaa tcggtcagtt catggaccag tatcctgagg | 1020 |

tgtttggcaa gccgcagtac

| | |
|---|---|
| cagaaaggcc cgggtcagga ggtgaaaacc gatgacaaat | 1080 |

catgggcaaa agcactgagc

| | |
|---|---|
| aagccagatg acgacggaat gacaccagcc agtatggagc | 1140 |

agttcaacaa agccaagggc

| | |
|---|---|
| atgatcaaaa ggcccatggc gggtgatacc ggcaacggca | 1200 |

acctgcaggc acgcggtgcc

| | |
|---|---|
| ggtggttctt cgctgggtat tgatgccatg atggccggtg | 1260 |

atgccattaa caatatggca

| | |
|---|---|
| cttggcaagc tgggcgcggc ttaagctt | 1288 |

The above nucleotide and amino acid sequences are disclosed are further described in U.S. Pat. No. 5,849,868 to Beer et al. and U.S. Pat. No. 5,776,889 to Wei et al., each of which is hereby incorporated by reference in its entirety.

Another hypersensitive response elicitor protein or polypeptide derived from Erwinia amylovora has an amino acid sequence corresponding to SEQ. ID. No. 5 as follows:

```
Met Ser Ile Leu Thr Leu Asn Asn Asn Thr Ser Ser
1 5 10
Ser Pro Gly Leu Phe Gln Ser Gly Gly Asp Asn Gly
                15 20
Leu Gly Gly His Asn Ala Asn Ser Ala Leu Gly Gln
25 30 35
Gln Pro Ile Asp Arg Gln Thr Ile Glu Gln Met Ala
                          40 45
Gln Leu Leu Ala Glu Leu Leu Lys Ser Leu Leu Ser
        50 55 60
Pro Gln Ser Gly Asn Ala Ala Thr Gly Ala Gly Gly
65 70
Asn Asp Gln Thr Thr Gly Val Gly Asn Ala Gly Gly
                75 80
Leu Asn Gly Arg Lys Gly Thr Ala Gly Thr Thr Pro
85 90 95
Gln Ser Asp Ser Gln Asn Met Leu Ser Glu Met Gly
                        100 105
Asn Asn Gly Leu Asp Gln Ala Ile Thr Pro Asp Gly
        110 115 120
Gln Gly Gly Gly Gln Ile Gly Asp Asn Pro Leu Leu
                                  125 130
Lys Ala Met Leu Lys Leu Ile Ala Arg Met Met Asp
                135 140
Gly Gln Ser Asp Gln Phe Gly Gln Pro Gly Thr Gly
145 150 155
Asn Asn Ser Ala Ser Ser Gly Thr Ser Ser Ser Gly
                        160 165
Gly Ser Pro Phe Asn Asp Leu Ser Gly Gly Lys Ala
        170 175 180
Pro Ser Gly Asn Ser Pro Ser Gly Asn Tyr Ser Pro
                                  185 190
Val Ser Thr Phe Ser Pro Pro Ser Thr Pro Thr Ser
                195 200
Pro Thr Ser Pro Leu Asp Phe Pro Ser Ser Pro Thr
205 210 215
Lys Ala Ala Gly Gly Ser Thr Pro Val Thr Asp His
                        220 225
Pro Asp Pro Val Gly Ser Ala Gly Ile Gly Ala Gly
        230 235 240
Asn Ser Val Ala Phe Thr Ser Ala Gly Ala Asn Gln
                                245 250
Thr Val Leu His Asp Thr Ile Thr Val Lys Ala Gly
                255 260
Gln Val Phe Asp Gly Lys Gly Gln Thr Phe Thr Ala
265 270 275
Gly Ser Glu Leu Gly Asp Gly Gly Gln Ser Glu Asn
                        280 285
Gln Lys Pro Leu Phe Ile Leu Glu Asp Gly Ala Ser
        290 295 300
Leu Lys Asn Val Thr Met Gly Asp Asp Gly Ala Asp
305 310
Gly Ile His Leu Tyr Gly Asp Ala Lys Ile Asp Asn
                315 320
Leu His Val Thr Asn Val Gly Glu Asp Ala Ile Thr
325 330 335
Val Lys Pro Asn Ser Ala Gly Lys Lys Ser His Val
                        340 345
Glu Ile Thr Asn Ser Ser Phe Glu His Ala Ser Asp
        350 355 360
Lys Ile Leu Gln Leu Asn Ala Asp Thr Asn Leu Ser
                                  365 370
Val Asp Asn Val Lys Ala Lys Asp Phe Gly Thr Phe
                375 380
Val Arg Thr Asn Gly Gly Gln Gln Gly Asn Trp Asp
385 390 395
Leu Asn Leu Ser His Ile Ser Ala Glu Asp Gly Lys
                        400 405
Phe Ser Phe Val Lys Ser Asp Ser Glu Gly Leu Asn
        410 415 420
Val Asn Thr Ser Asp Ile Ser Leu Gly Asp Val Glu
                                  425 430
Asn His Tyr Lys Val Pro Met Ser Ala Asn Leu Lys
                435 440
Val Ala Glu
445
```

This protein or polypeptide is acidic, rich in glycine and serine, and lacks cysteine. It is also heat stable, protease sensitive, and suppressed by inhibitors of plant metabolism. The protein or polypeptide of the present invention has a predicted molecular mass of ca. 45 kDa. The DNA molecule encoding this hypersensitive response elicitor protein or polypeptide has a nucleotide sequence corresponding to SEQ. ID. No. 6 as follows:

| | |
|---|---|
| atgtcaattc ttacgcttaa caacaatacc tcgtcctcgc | 60 |

cgggtctgtt ccagtccggg

| | |
|---|---|
| ggggacaacg ggcttggtgg tcataatgca aattctgcgt | 120 |

tggggcaaca acccatcgat

| | |
|---|---|
| cggcaaacca ttgagcaaat ggctcaatta ttggcggaac | 180 |

tgttaaagtc actgctatcg

| | |
|---|---|
| ccacaatcag gtaatgcggc aaccggagcc ggtggcaatg | 240 |

accagactac aggagttggt

| | |
|---|---|
| aacgctggcg gcctgaacgg acgaaaaggc acagcaggaa | 300 |

ccactccgca gtctgacagt

| | |
|---|---|
| cagaacatgc tgagtgagat gggcaacaac gggctggatc | 360 |

aggccatcac gcccgatggc

| | |
|---|---|
| cagggcggcg ggcagatcgg cgataatcct ttactgaaag | 420 |

ccatgctgaa gcttattgca

| | |
|---|---|
| cgcatgatgg acggccaaag cgatcagttt ggccaacctg | 480 |

gtacgggcaa caacagtgcc

| | |
|---|---|
| tcttccggta cttcttcatc tggcggttcc ccttttaacg | 540 |

atctatcagg ggggaaggcc

| | |
|---|---|
| ccttccggca actccccttc cggcaactac tctcccgtca | 600 |

gtaccttctc acccccatcc

| | |
|---|---|
| acgccaacgt cccctacctc accgcttgat ttcccttctt | 660 |

ctcccaccaa agcagccggg

| | |
|---|---|
| ggcagcacgc cggtaaccga tcatcctgac cctgttggta | 720 |

gcgcgggcat cggggccgga

| | |
|---|---|
| aattcggtgg ccttcaccag cgccggcgct aatcagacgg | 780 |

tgctgcatga caccattacc

| | |
|---|---|
| gtgaaagcgg gtcaggtgtt tgatggcaaa ggacaaacct | 840 |

tcaccgccgg ttcagaatta

| | |
|---|---|
| ggcgatggcg gccagtctga aaaccagaaa ccgctgttta | 900 |

tactggaaga cggtgccagc

| | |
|---|---|
| ctgaaaaacg tcaccatggg cgacgacggg gcggatggta | 960 |

ttcatcttta cggtgatgcc

| | |
|---|---|
| aaaatagaca atctgcacgt caccaacgtg ggtgaggacg | 1020 |

cgattaccgt taagccaaac

| | |
|---|---|
| agcgcgggca aaaaatccca cgttgaaatc actaacagtt | 1080 |

ccttcgagca cgcctctgac

| | |
|---|---|
| aagatcctgc agctgaatgc cgatactaac ctgagcgttg | 1140 |

acaacgtgaa ggccaaagac

| | |
|---|---|
| tttggtactt ttgtacgcac taacggcggt caacagggta | 1200 |

actgggatct gaatctgagc

| | |
|---|---|
| catatcagcg cagaagacgg taagttctcg ttcgttaaaa | 1260 |

gcgatagcga ggggctaaac

| | |
|---|---|
| gtcaatacca gtgatatctc actgggtgat gttgaaaacc | 1320 |

actacaaagt gccgatgtcc

| | |
|---|---|
| gccaacctga aggtggctga atga | 1344 |

The above nucleotide and amino acid sequences are disclosed and further described in U.S. Pat. No. 6,262,018 to Kim et al., which is hereby incorporated by reference in its entirety.

A hypersensitive response elicitor protein or polypeptide derived from Pseudomonas syringae has an amino acid sequence corresponding to SEQ. ID. No. 7 as follows:

```
Met Gln Ser Leu Ser Leu Asn Ser Ser Ser Leu Gln
1 5 10
Thr Pro Ala Met Ala Leu Val Leu Val Arg Pro Glu
                15 20
Ala Glu Thr Thr Gly Ser Thr Ser Ser Lys Ala Leu
25 30 35
Gln Glu Val Val Val Lys Leu Ala Glu Glu Leu Met
                        40 45
Arg Asn Gly Gln Leu Asp Asp Ser Ser Pro Leu Gly
        50 55 60
Lys Leu Leu Ala Lys Ser Met Ala Ala Asp Gly Lys
65 70
Ala Gly Gly Gly Ile Glu Asp Val Ile Ala Ala Leu
                75 80
Asp Lys Leu Ile His Glu Lys Leu Gly Asp Asn Phe
85 90 95
Gly Ala Ser Ala Asp Ser Ala Ser Gly Thr Gly Gln
                        100 105
Gln Asp Leu Met Thr Gln Val Leu Asn Gly Leu Ala
        110 115 120
Lys Ser Met Leu Asp Asp Leu Leu Thr Lys Gln Asp
                                125 130
Gly Gly Thr Ser Phe Ser Glu Asp Asp Met Pro Met
                135 140
Leu Asn Lys Ile Ala Gln Phe Met Asp Asp Asn Pro
145 150 155
Ala Gln Phe Pro Lys Pro Asp Ser Gly Ser Trp Val
                        160 165
Asn Glu Leu Lys Glu Asp Asn Phe Leu Asp Gly Asp
        170 175 180
Glu Thr Ala Ala Phe Arg Ser Ala Leu Asp Ile Ile
                                185 190
Gly Gln Gln Leu Gly Asn Gln Gln Ser Asp Ala Gly
                195 200
Ser Leu Ala Gly Thr Gly Gly Gly Leu Gly Thr Pro
205 210 215
Ser Ser Phe Ser Asn Asn Ser Ser Val Met Gly Asp
                        220 225
Pro Leu Ile Asp Ala Asn Thr Gly Pro Gly Asp Ser
        230 235 240
Gly Asn Thr Arg Gly Glu Ala Gly Gln Leu Ile Gly
                                245 250
Glu Leu Ile Asp Arg Gly Leu Gln Ser Val Leu Ala
                255 260
Gly Gly Gly Leu Gly Thr Pro Val Asn Thr Pro Gln
265 270 275
Thr Gly Thr Ser Ala Asn Gly Gly Gln Ser Ala Gln
                        280 285
Asp Leu Asp Gln Leu Leu Gly Gly Leu Leu Leu Lys
        290 295 300
Gly Leu Glu Ala Thr Leu Lys Asp Ala Gly Gln Thr
305 310
Gly Thr Asp Val Gln Ser Ser Ala Ala Gln Ile Ala
                315 320
Thr Leu Leu Val Ser Thr Leu Leu Gln Gly Thr Arg
325 330 335
Asn Gln Ala Ala Ala
                        340
```

This hypersensitive response elicitor protein or polypeptide has a molecular mass of 34-35 kDa. It is rich in glycine (about 13.5%) and lacks cysteine and tyrosine. Further information about the hypersensitive response elicitor derived from Pseudomonas syringae is found in He, S. Y., et al., “Pseudomonas syringae pv. syringae Harpin_{Pss}: a Protein that is Secreted via the Hrp Pathway and Elicits the Hypersensitive Response in Plants,” Cell 73:1255-1266 (1993), which is hereby incorporated by reference in its entirety. The DNA molecule encoding this hypersensitive response elicitor from Pseudomonas syringae has a nucleotide sequence corresponding to SEQ. ID. No. 8 as follows:

| | |
|---|---|
| atgcagagtc tcagtcttaa cagcagctcg ctgcaaaccc | 60 |

cggcaatggc ccttgtcctg

| | |
|---|---|
| gtacgtcctg aagccgagac gactggcagt acgtcgagca | 120 |

aqgcgcttca ggaagttgtc

| | |
|---|---|
| gtgaagctgg ccgaggaact gatgcgcaat ggtcaactcg | 180 |

acgacagctc gccattggga

| | |
|---|---|
| aaactgttgg ccaagtcgat ggccgcagat ggcaaggcgg | 240 |

gcggcggtat tgaggatgtc

| | |
|---|---|
| atcgctgcgc tggacaagct gatccatgaa aagctcggtg | 300 |

acaacttcgg cgcgtctgcg

| | |
|---|---|
| gacagcgcct cgggtaccgg acagcaggac ctgatgactc | 360 |

aggtgctcaa tggcctggcc

| | |
|---|---|
| aagtcgatgc tcgatgatct tctgaccaag caggatggcg | 420 |

ggacaagctt ctccgaagac

| | |
|---|---|
| gatatgccga tgctgaacaa gatcgcgcag ttcatggatg | 480 |

acaatcccgc acagtttccc

| | |
|---|---|
| aagccggact cgggctcctg ggtgaacgaa ctcaaggaag | 540 |

acaacttcct tgatggcgac

| | |
|---|---|
| gaaacggctg cgttccgttc ggcactcgac atcattggcc | 600 |

agcaactggg taatcagcag

| | |
|---|---|
| agtgacgctg gcagtctggc agggacgggt ggaggtctgg | 660 |

gcactccgag cagtttttcc

| | |
|---|---|
| aacaactcgt ccgtgatggg tgatccgctg atcgacgcca | 720 |

ataccggtcc cggtgacagc

| | |
|---|---|
| ggcaataccc gtggtgaagc ggggcaactg atcggcgagc | 780 |

ttatcgaccg tggcctgcaa

| | |
|---|---|
| tcggtattgg ccggtggtgg actgggcaca cccgtaaaca | 840 |

ccccgcagac cggtacgtcg

| | |
|---|---|
| gcgaatggcg gacagtccgc tcaggatctt gatcagttgc | 900 |

tgggcggctt gctgctcaag

| | |
|---|---|
| ggcctggagg caacgctcaa ggatgccggg caaacaggca | 960 |

ccgacgtgca gtcgagcgct

| | |
|---|---|
| gcgcaaatcg ccaccttgct ggtcagtacg ctgctgcaag | 1020 |

gcacccgcaa tcaggctgca

| | |
|---|---|
| gcctga | 1026 |

The above nucleotide and amino acid sequences are disclosed and further described in U.S. Pat. No. 5,708,139 to Collmer et al. and U.S. Pat. No. 5,776,889 to Wei et al., each of which is hereby incorporated by reference in its entirety.

Another hypersensitive response elicitor protein or polypeptide derived from Pseudomonas syringae has an amino acid sequence corresponding to SEQ. ID. No. 9 as follows:

```
Met Ser Ile Gly Ile Thr Pro Arg Pro Gln Gln Thr
1 5 10
Thr Thr Pro Leu Asp Phe Ser Ala Leu Ser Gly Lys
                15 20
Ser Pro Gln Pro Asn Thr Phe Gly Glu Gln Asn Thr
25 30 35
Gln Gln Ala Ile Asp Pro Ser Ala Leu Leu Phe Gly
                        40 45
Ser Asp Thr Gln Lys Asp Val Asn Phe Gly Thr Pro
        50 55 60
Asp Ser Thr Val Gln Asn Pro Gln Asp Ala Ser Lys
65 70
Pro Asn Asp Ser Gln Ser Asn Ile Ala Lys Leu Ile
                75 80
Ser Ala Leu Ile Met Ser Leu Leu Gln Met Leu Thr
85 90 95
Asn Ser Asn Lys Lys Gln Asp Thr Asn Gln Glu Gln
                        100 105
Pro Asp Ser Gln Ala Pro Phe Gln Asn Asn Gly Gly
        110 115 120
Leu Gly Thr Pro Ser Ala Asp Ser Gly Gly Gly Gly
                                125 130
Thr Pro Asp Ala Thr Gly Gly Gly Gly Gly Asp Thr
                135 140
Pro Ser Ala Thr Gly Gly Gly Gly Gly Asp Thr Pro
145 150 155
Thr Ala Thr Gly Gly Gly Gly Ser Gly Gly Gly Gly
                        160 165
Thr Pro Thr Ala Thr Gly Gly Gly Ser Gly Gly Thr
        170 175 180
Pro Thr Ala Thr Gly Gly Gly Glu Gly Gly Val Thr
                                185 190
Pro Gln Ile Thr Pro Gln Leu Ala Asn Pro Asn Arg
                195 200
Thr Ser Gly Thr Gly Ser Val Ser Asp Thr Ala Gly
205 210 215
Ser Thr Glu Gln Ala Gly Lys Ile Asn Val Val Lys
                        220 225
Asp Thr Ile Lys Val Gly Ala Gly Glu Val Phe Asp
        230 235 240
Gly His Gly Ala Thr Phe Thr Ala Asp Lys Ser Met
                                245 250
Gly Asn Gly Asp Gln Gly Glu Asn Gln Lys Pro Met
                255 260
Phe Glu Leu Ala Glu Gly Ala Thr Leu Lys Asn Val
265 270 275
Asn Leu Gly Glu Asn Glu Val Asp Gly Ile His Val
                        280 285
Lys Ala Lys Asn Ala Gln Glu Val Thr Ile Asp Asn
        290 295 300
Val His Ala Gln Asn Val Gly Glu Asp Leu Ile Thr
305 310
Val Lys Gly Glu Gly Gly Ala Ala Val Thr Asn Leu
                315 320
Asn Ile Lys Asn Ser Ser Ala Lys Gly Ala Asp Asp
325 330 335
Lys Val Val Gln Leu Asn Ala Asn Thr His Leu Lys
                        340 345
Ile Asp Asn Phe Lys Ala Asp Asp Phe Gly Thr Met
        350 355 360
Val Arg Thr Asn Gly Gly Lys Gln Phe Asp Asp Met
                                365 370
Ser Ile Glu Leu Asn Gly Ile Glu Ala Asn His Gly
                375 380
Lys Phe Ala Leu Val Lys Ser Asp Ser Asp Asp Leu
385 390 395
Lys Leu Ala Thr Gly Asn Ile Ala Met Thr Asp Val
                        400 405
Lys His Ala Tyr Asp Lys Thr Gln Ala Ser Thr Gln
        410 415 420
His Thr Glu Leu
```

This protein or polypeptide is acidic, glycine-rich, lacks cysteine, and is deficient in aromatic amino acids. The DNA molecule encoding this hypersensitive response elicitor from Pseudomonas syringae has a nucleotide sequence corresponding to SEQ. ID. No. 10 as follows:

| | |
|---|---|
| tccacttcgc tgattttgaa attggcagat tcatagaaac | 60 |

gttcaggtgt ggaaatcagg

| | |
|---|---|
| ctgagtgcgc agatttcgtt gataagggtg tggtactggt | 120 |

cattgttggt catttcaagg

| | |
|---|---|
| cctctgagtg cggtgcggag caataccagt cttcctgctg | 180 |

gcgtgtgcac actgagtcgc

| | |
|---|---|
| aggcataggc atttcagttc cttgcgttgg ttgggcatat | 240 |

aaaaaaagga acttttaaaa

| | |
|---|---|
| acagtgcaat gagatgccgg caaaacggga accggtcgct | 300 |

gcgctttgcc actcacttcg

| | |
|---|---|
| agcaagctca accccaaaca tccacatccc tatcgaacgg | 360 |

acagcgatac ggccacttgc

| | |
|---|---|
| tctggtaaac cctggagctg gcgtcggtcc aattgcccac | 420 |

ttagcgaggt aacgcagcat

| | |
|---|---|
| gagcatcggc atcacacccc ggccgcaaca gaccaccacg | 480 |

ccactcgatt tttcggcgct

| | |
|---|---|
| aagcggcaag agtcctcaac caaacacgtt cggcgagcag | 540 |

aacactcagc aagcgatcga

| | |
|---|---|
| cccgagtgca ctgttgttcg gcagcgacac acagaaagac | 600 |

gtcaacttcg gcacgcccga

| | |
|---|---|
| cagcaccgtc cagaatccgc aggacgccag caagcccaac | 660 |

gacagccagt ccaacatcgc

| | |
|---|---|
| taaattgatc agtgcattga tcatgtcgtt gctgcagatg | 720 |

ctcaccaact ccaataaaaa

| | |
|---|---|
| gcaggacacc aatcaggaac agcctgatag ccaggctcct | 780 |

ttccagaaca acggcgggct

| | |
|---|---|
| cggtacaccg tcggccgata gcgggggcgg cggtacaccg | 840 |

gatgcgacag gtggcggcgg

| | |
|---|---|
| cggtgatacg ccaagcgcaa caggcggtgg cggcggtgat | 900 |

actccgaccg caacaggcgg

| | |
|---|---|
| tggcggcagc ggtggcggcg gcacacccac tgcaacaggt | 960 |

ggcggcagcg gtggcacacc

| | |
|---|---|
| cactgcaaca ggcggtggcg agggtggcgt aacaccgcaa | 1020 |

atcactccgc agttggccaa

| | |
|---|---|
| ccctaaccgt acctcaggta ctggctcggt gtcggacacc | 1080 |

gcaggttcta ccgagcaagc

| | |
|---|---|
| cggcaagatc aatgtggtga aagacaccat caaggtcggc | 1140 |

gctggcgaag tctttgacgg

| | |
|---|---|
| ccacggcgca accttcactg ccgacaaatc tatgggtaac | 1200 |

ggagaccagg gcgaaaatca

| | |
|---|---|
| gaagcccatg ttcgagctgg ctgaaggcgc tacgttgaag | 1260 |

aatgtgaacc tgggtgagaa

| | |
|---|---|
| cgaggtcgat ggcatccacg tgaaagccaa aaacgctcag | 1320 |

gaagtcacca ttgacaacgt

| | |
|---|---|
| gcatgcccag aacgtcggtg aagacctgat tacggtcaaa | 1380 |

ggcgagggag gcgcagcggt

| | |
|---|---|
| cactaatctg aacatcaaga acagcagtgc caaaggtgca | 1440 |

gacgacaagg ttgtccagct

| | |
|---|---|
| caacgccaac actcacttga aaatcgacaa cttcaaggcc | 1500 |

gacgatttcg gcacgatggt

| | |
|---|---|
| tcgcaccaac ggtggcaagc agtttgatga catgagcatc | 1560 |

gagctgaacg gcatcgaagc

| | |
|---|---|
| taaccacggc aagttcgccc tggtgaaaag cgacagtgac | 1620 |

gatctgaagc tggcaacggg

| | |
|---|---|
| caacatcgcc atgaccgacg tcaaacacgc ctacgataaa | 1680 |

acccaggcat cgacccaaca

| | |
|---|---|
| caccgagctt tgaatccaga caagtagctt gaaaaaaggg | 1729 |

ggtggactc

The above nucleotide and amino acid sequences are disclosed and further described in U.S. Pat. No. 6,172,184 to Collmer et al., which is hereby incorporated by reference in its entirety.

A hypersensitive response elicitor protein or polypeptide derived from Ralstonia solanacearum has an amino acid sequence corresponding to SEQ. ID. No. 11 as follows:

```
Met Ser Val Gly Asn Ile Gln Ser Pro Ser Asn Leu
1 5 10
Pro Gly Leu Gln Asn Leu Asn Leu Asn Thr Asn Thr
                15 20
Asn Ser Gln Gln Ser Gly Gln Ser Val Gln Asp Leu
25 30 35
Ile Lys Gln Val Glu Lys Asp Ile Leu Asn Ile Ile
                        40 45
Ala Ala Leu Val Gln Lys Ala Ala Gln Ser Ala Gly
        50 55 60
Gly Asn Thr Gly Asn Thr Gly Asn Ala Pro Ala Lys
                                65 70
Asp Gly Asn Ala Asn Ala Gly Ala Asn Asp Pro Ser
                75 80
Lys Asn Asp Pro Ser Lys Ser Gln Ala Pro Gln Ser
85 90 95
Ala Asn Lys Thr Gly Asn Val Asp Asp Ala Asn Asn
                        100 105
Gln Asp Pro Met Gln Ala Leu Met Gln Leu Leu Glu
        110 115 120
Asp Leu Val Lys Leu Leu Lys Ala Ala Leu His Met
                                125 130
Gln Gln Pro Gly Gly Asn Asp Lys Gly Asn Gly Val
                135 140
Gly Gly Ala Asn Gly Ala Lys Gly Ala Gly Gly Gln
145 150 155
Gly Gly Leu Ala Glu Ala Leu Gln Glu Ile Glu Gln
                        160 165
Ile Leu Ala Gln Leu Gly Gly Gly Gly Ala Gly Ala
        170 175 180
Gly Gly Ala Gly Gly Gly Val Gly Gly Ala Gly Gly
                        185 190
Ala Asp Gly Gly Ser Gly Ala Gly Gly Ala Gly Gly
                195 200
Ala Asn Gly Ala Asp Gly Gly Asn Gly Val Asn Gly
205 210 215
Asn Gln Ala Asn Gly Pro Gln Asn Ala Gly Asp Val
                        220 225
Asn Gly Ala Asn Gly Ala Asp Asp Gly Ser Glu Asp
        230 235 240
Gln Gly Gly Leu Thr Gly Val Leu Gln Lys Leu Met
                                245 250
Lys Ile Leu Asn Ala Leu Val Gln Met Met Gln Gln
                255 260
Gly Gly Leu Gly Gly Gly Asn Gln Ala Gln Gly Gly
265 270 275
Ser Lys Gly Ala Gly Asn Ala Ser Pro Ala Ser Gly
                        280 285
Ala Asn Pro Gly Ala Asn Gln Pro Gly Ser Ala Asp
        290 295 300
Asp Gln Ser Ser Gly Gln Asn Asn Leu Gln Ser Gln
                                305 310
Ile Met Asp Val Val Lys Glu Val Val Gln Ile Leu
                315 320
Gln Gln Met Leu Ala Ala Gln Asn Gly Gly Ser Gln
325 330 335
Gln Ser Thr Ser Thr Gln Pro Met
                        340
```

Further information regarding this hypersensitive response elicitor protein or polypeptide derived from Ralstonia solanacearum is set forth in Arlat, M., et al., “PopA1, a Protein which Induces a Hypersensitive-like Response in Specific Petunia Genotypes, is Secreted via the Hrp Pathway of Pseudomonas solanacearum,” EMBO J. 13:543-533 (1994), which is hereby incorporated by reference in its entirety. It is encoded by a DNA molecule from Ralstonia solanacearum having a nucleotide sequence corresponding SEQ. ID. No. 12 as follows:

| | |
|---|---|
| atgtcagtcg gaaacatcca gagcccgtcg aacctcccgg | 60 |

gtctgcagaa cctgaacctc

| | |
|---|---|
| aacaccaaca ccaacagcca gcaatcgggc cagtccgtgc | 120 |

aagacctgat caagcaggtc

| | |
|---|---|
| gagaaggaca tcctcaacat catcgcagcc ctcgtgcaga | 180 |

aggccgcaca gtcggcgggc

| | |
|---|---|
| ggcaacaccg gtaacaccgg caacgcgccg gcgaaggacg | 240 |

gcaatgccaa cgcgggcgcc

| | |
|---|---|
| aacgacccga gcaagaacga cccgagcaag agccaggctc | 300 |

cgcagtcggc caacaagacc

| | |
|---|---|
| ggcaacgtcg acgacgccaa caaccaggat ccgatgcaag | 360 |

cgctgatgca gctgctggaa

| | |
|---|---|
| gacctggtga agctgctgaa ggcggccctg cacatgcagc | 420 |

agcccggcgg caatgacaag

| | |
|---|---|
| ggcaacggcg tgggcggtgc caacggcgcc aagggtgccg | 480 |

gcggccaggg cggcctggcc

| | |
|---|---|
| gaagcgctgc aggagatcga gcagatcctc gcccagctcg | 540 |

gcggcggcgg tgctggcgcc

| | |
|---|---|
| ggcggcgcgg gtggcggtgt cggcggtgct ggtggcgcgg | 600 |

atggcggctc cggtgcgggt

| | |
|---|---|
| ggcgcaggcg gtgcgaacgg cgccgacggc ggcaatggcg | 660 |

tgaacggcaa ccaggcgaac

| | |
|---|---|
| ggcccgcaga acgcaggcga tgtcaacggt gccaacggcg | 720 |

cggatgacgg cagcgaagac

| | |
|---|---|
| cagggcggcc tcaccggcgt gctgcaaaag ctgatgaaga | 780 |

tcctgaacgc gctggtgcag

| | |
|---|---|
| atgatgcagc aaggcggcct cggcggcggc aaccaggcgc | 840 |

agggcggctc gaagggtgcc

| | |
|---|---|
| ggcaacgcct cgccggcttc cggcgcgaac ccgggcgcga | 900 |

accagcccgg ttcggcggat

| | |
|---|---|
| gatcaatcgt ccggccagaa caatctgcaa tcccagatca | 960 |

tggatgtggt gaaggaggtc

| | |
|---|---|
| gtccagatcc tgcagcagat gctggcggcg cagaacggcg | 1020 |

gcagccagca gtccacctcg

| | |
|---|---|
| acgcagccga tgtaa | 1035 |

The above nucleotide and amino acid sequences are disclosed and further described in U.S. Pat. No. 5,776,889 to Wei et al., which is hereby incorporated by reference in its entirety.

A hypersensitive response elicitor protein or polypeptide derived from Xanthomonas campestris has an amino acid sequence corresponding to SEQ. ID. No. 13 as follows:

```
Met Asp Ser Ile Gly Asn Asn Phe Ser Asn Ile Gly
    1 5 10
Asn Leu Gln Thr Met Gly Ile Gly Pro Gln Gln His
                  15 20
Glu Asp Ser Ser Gln Gln Ser Pro Ser Ala Gly Ser
  25 30 35
Glu Gln Gln Leu Asp Gln Leu Leu Ala Met Phe Ile
                          40 45
Met Met Met Leu Gln Gln Ser Gln Gly Ser Asp Ala
          50 55 60
Asn Gln Glu Cys Gly Asn Glu Gln Pro Gln Asn Gly
                                  65 70
Gln Gln Glu Gly Leu Ser Pro Leu Thr Gln Met Leu
                  75 80
Met Gln Ile Val Met Gln Leu Met Gln Asn Gln Gly
  85 90 95
Gly Ala Gly Met Gly Gly Gly Gly Ser Val Asn Ser
                        100 105
Ser Leu Gly Gly Asn Ala
        110
```

This hypersensitive response elicitor protein has an estimated molecular mass of about 12 kDa based on the deduced amino acid sequence, which is consistent with the molecular mass of about 14 kDa as detected by SDS-PAGE. It is encoded by a DNA molecule from Xanthomonas campestris having a nucleotide sequence corresponding SEQ. ID. No. 14 as follows:

| | |
|---|---|
| atggactcta tcggaaacaa cttttcgaat atcggcaacc | 60 |

tgcagacgat gggcatcggg

| | |
|---|---|
| cctcagcaac acgaggactc cagccagcag tcgccttcgg | 120 |

ctggctccga gcagcagctg

| | |
|---|---|
| gatcagttgc tcgccatgtt catcatgatg atgctgcaac | 180 |

agagccaggg cagcgatgca

| | |
|---|---|
| aatcaggagt gtggcaacga acaaccgcag aacggtcaac | 240 |

aggaaggcct gagtccgttg

| | |
|---|---|
| acgcagatgc tgatgcagat cgtgatgcag ctgatgcaga | 300 |

accagggcgg cgccggcatg

| | |
|---|---|
| ggcggtggcg gttcggtcaa cagcagcctg ggcggcaacg cc | 342 |

The above protein and nucleic acid molecule are further described in U.S. patent application Ser. No. 09/412,452 to Wei et al., filed Apr. 9, 2001, which is hereby incorporated by reference in its entirety.

Other embodiments of the present invention include, but are not limited to, use of hypersensitive response elicitor proteins or polypeptides derived from Erwinia carotovora and Erwinia stewartii. Isolation of an Erwinia carotovora hypersensitive response elicitor protein or polypeptide is described in Cui, et al., “The RsmA Mutants of Erwinia carotovora subsp. carotovora Strain Ecc71 Overexpress hrpN_{Ecc} and Elicit a Hypersensitive Reaction-like Response in Tobacco Leaves,” MPMI, 9(7):565-73 (1996), which is hereby incorporated by reference in its entirety. A hypersensitive response elicitor protein or polypeptide of Erwinia stewartii is set forth in Ahmad, et al., “Harpin is Not Necessary for the Pathogenicity of Erwinia stewartii on Maize,” 8th Int'l. Cong. Molec. Plant-Microbe Interact., Jul. 14-19, 1996 and Ahmad, et al., “Harpin is Not Necessary for the Pathogenicity of Erwinia stewartii on Maize,” Ann. Mtg. Am. Phytopath. Soc., Jul. 27-31, 1996, each of which is hereby incorporated by reference in its entirety.

Hypersensitive response elicitor proteins or polypeptides from various Phytophthora species are described in Kaman, et al., “Extracellular Protein Elicitors from Phytophthora: Most Specificity and Induction of Resistance to Bacterial and Fungal Phytopathogens,” Molec. Plant-Microbe Interact., 6(1):15-25 (1993); Ricci, et al., “Structure and Activity of Proteins from Pathogenic Fungi Phytophthora Eliciting Necrosis and Acquired Resistance in Tobacco,” Eur. J. Biochem., 183:555-63 (1989); Ricci, et al., “Differential Production of Parasiticein, and Elicitor of Necrosis and Resistance in Tobacco, by Isolates of Phytophthora parasitica,” Plant Path. 41:298-307 (1992); Baillreul, et al., “A New Elicitor of the Hypersensitive Response in Tobacco: A Fungal Glycoprotein Elicits Cell Death, Expression of Defense Genes, Production of Salicylic Acid, and Induction of Systemic Acquired Resistance,” Plant J., 8(4):551-60 (1995), and Bonnet, et al., “Acquired Resistance Triggered by Elicitors in Tobacco and Other Plants,” Eur. J. Plant Path., 102:181-92 (1996), each of which is hereby incorporated by reference in its entirety.

Another hypersensitive response elicitor protein or polypeptide which can be used in accordance with the present invention is derived from Clavibacter michiganensis subsp. sepedonicus and is described in U.S. patent application Ser. No. 09/136,625 to Beer et al., filed Aug. 19, 1998, which is hereby incorporated by reference in its entirety.

Fragments of the above hypersensitive response elicitor proteins or polypeptides as well as fragments of full length elicitors from other pathogens can also be used according to the present invention.

Suitable fragments can be produced by several means. Subclones of the gene encoding a known elicitor protein can be produced using conventional molecular genetic manipulation for subcloning gene fragments, such as described by Sambrook et al., Molecular Cloning: A Laboratory Manual, Cold Springs Laboratory, Cold Springs Harbor, N.Y. (1989), and Ausubel et al. (ed.), Current Protocols in Molecular Biology, John Wiley & Sons (New York, N.Y.) (1999 and preceding editions), each of which is hereby incorporated by reference in its entirety. The subclones then are expressed in vitro or in vivo in bacterial cells to yield a smaller protein or polypeptide that can be tested for elicitor activity, e.g., using procedures set forth in Wei, Z-M., et al., Science 257: 85-88 (1992), which is hereby incorporated by reference in its entirety.

In another approach, based on knowledge of the primary structure of the protein, fragments of the elicitor protein gene may be synthesized using the PCR technique together with specific sets of primers chosen to represent particular portions of the protein. Erlich, H. A., et al., “Recent Advances in the Polymerase Chain Reaction,” Science 252:1643-51 (1991), which is hereby incorporated by reference in its entirety. These can then be cloned into an appropriate vector for expression of a truncated protein or polypeptide from bacterial cells as described above.

As an alternative, fragments of an elicitor protein can be produced by digestion of a full-length elicitor protein with proteolytic enzymes like chymotrypsin or Staphylococcus proteinase A, or trypsin. Different proteolytic enzymes are likely to cleave elicitor proteins at different sites based on the amino acid sequence of the elicitor protein. Some of the fragments that result from proteolysis may be active elicitors of resistance.

Chemical synthesis can also be used to make suitable fragments. Such a synthesis is carried out using known amino acid sequences for the elicitor being produced. Alternatively, subjecting a full length elicitor to high temperatures and pressures will produce fragments. These fragments can then be separated by conventional procedures (e.g., chromatography, SDS-PAGE).

An example of suitable fragments of a hypersensitive response elicitor which elicit a hypersensitive response are fragments of the Erwinia amylovora hypersensitive response elicitor protein or polypeptide of SEQ. ID. No. 3. The fragments can be a C-terminal fragment of the amino acid sequence of SEQ. ID. No. 3, an N-terminal fragment of the amino acid sequence of SEQ. ID. No. 3, or an internal fragment of the amino acid sequence of SEQ. ID. No. 3. The C-terminal fragment of the amino acid sequence of SEQ. ID. No. 3 can span amino acids 105 and 403 of SEQ. ID. No. 3. The N-terminal fragment of the amino acid sequence of SEQ. ID. No. 3 can span the following amino acids of SEQ. ID. No. 3: 1 and 98, 1 and 104, 1 and 122, 1 and 168, 1 and 218, 1 and 266, 1 and 342, 1 and 321, and 1 and 372. The internal fragment of the amino acid sequence of SEQ. ID. No. 3 can span the following amino acids of SEQ. ID. No. 3: 76 and 209, 105 and 209, 99 and 209, 137 and 204, 137 and 200, 109 and 204, 109 and 200, 137 and 180, and 105 and 180. DNA molecules encoding these fragments can also be utilized in a chimeric gene of the present invention.

Variants may also (or alternatively) be modified by, for example, the deletion or addition of amino acids that have minimal influence on the properties, secondary structure and hydropathic nature of the polypeptide. For example, a polypeptide may be conjugated to a signal (or leader) sequence at the N-terminal end of the protein which co-translationally or post-translationally directs transfer of the protein. The polypeptide may also be conjugated to a linker or other sequence for ease of synthesis, purification, or identification of the polypeptide.

The hypersensitive response elicitor proteins or polypeptides used in accordance with the present invention are preferably produced in purified form (preferably at least about 80%, more preferably 90%, pure) by conventional techniques. Typically, the protein or polypeptide of the present invention is produced but not secreted into growth medium. In such cases, to isolate the protein, the host cell (e.g., E. coli) carrying a recombinant plasmid is propagated, lysed by sonication, heat, or chemical treatment, and the homogenate is centrifuged to remove bacterial debris. The supernatant is then subjected to sequential ammonium sulfate precipitation. The fraction containing the hypersensitive response elicitor protein or polypeptide of interest is subjected to gel filtration in an appropriately sized dextran or polyacrylamide column to separate the proteins. If necessary, the protein fraction may be further purified by HPLC. Alternatively, the protein or polypeptide of the present invention is secreted into the growth medium of recombinant host cells (discussed infra) and removed therefrom.

One particular hypersensitive response elicitor protein, known as harpin_{Ea}, is commercially available from Eden Bioscience Corporation (Bothell, Wash.) under the name of Messenger®. Messenger® contains 3% by weight of harpin_{Ea} as the active ingredient and 97% by weight inert ingredients. Harpin_{Ea} is one type of hypersensitive response elicitor protein from Erwinia amylovora, identified herein by SEQ. ID. No. 3.

Other hypersensitive response elicitors can be readily identified by isolating putative protein or polypeptide candidates and testing them for elicitor activity as described, for example, in Wei, Z-M., et al., “Harpin, Elicitor of the Hypersensitive Response Produced by the Plant Pathogen Erwinia amylovora,” Science 257:85-88 (1992), which is hereby incorporated by reference in its entirety. Cell-free preparations from culture supernatants can be tested for elicitor activity (i.e., local necrosis) by using them to infiltrate appropriate plant tissues. Once identified, DNA molecules encoding a hypersensitive response elicitor can be isolated using standard techniques known to those skilled in the art.

DNA molecules encoding other hypersensitive response elicitor proteins or polypeptides can also be identified by determining whether such DNA molecules hybridizes under stringent conditions to a DNA molecule having the nucleotide sequence of SEQ. ID. Nos. 2, 4, 6, 8, 10, 12, or 14. An example of suitable stringency conditions is when hybridization is carried out at a temperature of about 37° C. using a hybridization medium that includes 0.9M sodium citrate (“SSC”) buffer, followed by washing with 0.2×SSC buffer at 37° C. Higher stringency can readily be attained by increasing the temperature for either hybridization or washing conditions or increasing the sodium concentration of the hybridization or wash medium. Nonspecific binding may also be controlled using any one of a number of known techniques such as, for example, blocking the membrane with protein-containing solutions, addition of heterologous RNA, DNA, and SDS to the hybridization buffer, and treatment with RNase. Wash conditions are typically performed at or below stringency. Exemplary high stringency conditions include carrying out hybridization at a temperature of about 42° C. to about 65° C. for up to about 20 hours in a hybridization medium containing 1M NaCl, 50 mM Tris-HCl, pH 7.4, 10 mM EDTA, 0.1% sodium dodecyl sulfate (SDS), 0.2% ficoll, 0.2% polyvinylpyrrolidone, 0.2% bovine serum albumin, and 50 μg/ml E. coli DNA, followed by washing carried out at between about 42° C. to about 65° C. in a 0.2×SSC buffer.

The DNA molecule encoding the hypersensitive response elicitor polypeptide or protein can be incorporated in cells using conventional recombinant DNA technology. Generally, this involves inserting the DNA molecule into an expression system to which the DNA molecule is heterologous (i.e. not normally present). The heterologous DNA molecule is inserted into the expression system or vector in proper sense orientation and correct reading frame. The vector contains the necessary elements for the transcription and translation of the inserted protein-coding sequences.

U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture.

Recombinant genes may also be introduced into viruses, such as vaccina virus. Recombinant viruses can be generated by transfection of plasmids into cells infected with virus.

Suitable vectors include, but are not limited to, the following viral vectors such as lambda vector system gt11, gt WES.tB, Charon 4, and plasmid vectors such as pBR322, pBR325, pACYC177, pACYC1084, pUC8, pUC9, pUC18, pUC19, pLG339, pR290, pKC37, pKC101, SV 40, pBluescript II SK +/− or KS +/− (see “Stratagene Cloning Systems” Catalog (1993) from Stratagene, La Jolla, Calif., which is hereby incorporated by reference in its entirety), pQE, pIH821, pGEX, pET series (see F. W. Studier et. al., “Use of T7 RNA Polymerase to Direct Expression of Cloned Genes,” Gene Expression Technology vol. 185 (1990), which is hereby incorporated by reference in its entirety), and any derivatives thereof. Recombinant molecules can be introduced into cells via transformation, particularly transduction, conjugation, mobilization, or electroporation. The DNA sequences are cloned into the vector using standard cloning procedures in the art, as described by Sambrook et al., Molecular Cloning: A Laboratory Manual, Cold Springs Laboratory, Cold Springs Harbor, N.Y. (1989), which is hereby incorporated by reference in its entirety.

A variety of host-vector systems may be utilized to express the protein-encoding sequence(s). Primarily, the vector system must be compatible with the host cell used. Host-vector systems include but are not limited to the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria. The expression elements of these vectors vary in their strength and specificities. Depending upon the host-vector system utilized, any one of a number of suitable transcription and translation elements can be used.

Different genetic signals and processing events control many levels of gene expression (e.g., DNA transcription and messenger RNA (mRNA) translation).

Transcription of DNA is dependent upon the presence of a promoter which is a DNA sequence that directs the binding of RNA polymerase and thereby promotes mRNA synthesis. The DNA sequences of eukaryotic promoters differ from those of prokaryotic promoters. Furthermore, eukaryotic promoters and accompanying genetic signals may not be recognized in or may not function in a prokaryotic system, and, further, prokaryotic promoters are not recognized and do not function in eukaryotic cells.

Similarly, translation of mRNA in prokaryotes depends upon the presence of the proper prokaryotic signals which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression, see Roberts and Lauer, Methods in Enzymology, 68:473 (1979), which is hereby incorporated by reference in its entirety.

Promoters vary in their “strength” (i.e. their ability to promote transcription). For the purposes of expressing a cloned gene, it is desirable to use strong promoters in order to obtain a high level of transcription and, hence, expression of the gene. Depending upon the host cell system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Bacterial host cell strains and expression vectors may be chosen which inhibit the action of the promoter unless specifically induced. In certain operations, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Specific initiation signals are also required for efficient gene transcription and translation in prokaryotic cells. These transcription and translation initiation signals may vary in “strength” as measured by the quantity of gene specific messenger RNA and protein synthesized, respectively. The DNA expression vector, which contains a promoter, may also contain any combination of various “strong” transcription and/or translation initiation signals. For instance, efficient translation in E. coli requires an SD sequence about 7-9 bases 5′ to the initiation codon (“ATG”) to provide a ribosome binding site. Thus, any SD-ATG combination that can be utilized by host cell ribosomes may be employed. Such combinations include but are not limited to the SD-ATG combination from the cro gene or the N gene of coliphage lambda, or from the E. coli tryptophan E, D, C, B or A genes. Additionally, any SD-ATG combination produced by recombinant DNA or other techniques involving incorporation of synthetic nucleotides may be used.

Once the isolated DNA molecule encoding the hypersensitive response elicitor polypeptide or protein has been cloned into an expression system, it is ready to be incorporated into a host cell. Such incorporation can be carried out by the various forms of transformation noted above, depending upon the vector/host cell system. Suitable host cells include, but are not limited to, bacteria, virus, yeast, mammalian cells, insect, plant, and the like.

Because it is desirable for recombinant host cells to secrete the hypersensitive response elicitor protein or polypeptide, it is preferable that the host cell also be transformed with a type III secretion system in accordance with Ham et al., “A Cloned Erwinia chrysanthemi Hrp (Type III Protein Secretion) System Functions in Escherichia coli to Deliver Pseudomonas syringae Avr Signals to Plant Cells and Secrete Avr Proteins in Culture,” Microbiol. 95:10206-10211 (1998), which is hereby incorporated by reference in its entirety.

Isolation of the hypersensitive response elicitor protein or polypeptide from the host cell or growth medium can be carried out as described above.

The methods of the present invention can be performed by treating the ornamental plant or a cutting removed therefrom.

Before removal of a cutting, suitable application methods include, without limitation, high or low pressure spraying of the entire plant. After removal of a cutting, suitable application methods include, without limitation, low or high pressure spraying, coating, or immersion. Other suitable application procedures (both pre- and post-cutting) can be envisioned by those skilled in the art provided they are able to effect contact of the hypersensitive response elicitor protein or polypeptide with the cutting. Once treated, the cuttings can be handled, packed, shipped, and processed using conventional procedures to deliver the cuttings to distributors or end-consumers.

The hypersensitive response elicitor polypeptide or protein can be applied to cuttings in accordance with the present invention alone or in a mixture with other materials. Alternatively, the hypersensitive response elicitor polypeptide or protein can be applied separately to cuttings with other materials being applied at different times.

A composition suitable for treating ornamental plants or cuttings therefrom in accordance with the application embodiment of the present invention contains an isolated hypersensitive response elicitor polypeptide or protein in a carrier. Suitable carriers include water, aqueous solutions, slurries, or dry powders. The composition preferably contains greater than about 500 nM hypersensitive response elicitor polypeptide or protein, although greater or lesser amounts of the hypersensitive response elicitor polypeptide or protein depending on the rate of composition application and efficacy of different hypersensitive response elicitor proteins or polypeptides.

Although not required, this composition may contain additional additives including fertilizer, insecticide, fungicide, nematacide, and mixtures thereof. Suitable fertilizers include (NH₄)₂NO₃. An example of a suitable insecticide is Malathion. Useful fungicides include Captan.

Other suitable additives include buffering agents, wetting agents, coating agents, and ripening agents. These materials can be used either to facilitate the process of the present invention or to provide additive benefits to inhibit desiccation or promote flowering.

As indicated above, one embodiment of the present invention involves treating ornamental plants or their cuttings with an isolated hypersensitive response elicitor protein or polypeptide. The hypersensitive response elicitor protein or polypeptide can be isolated from its natural source (e.g., Erwinia amylovora, Pseudomonas syringae, etc.) or from recombinant source transformed with a DNA molecule encoding the protein or polypeptide.

Another aspect of the present invention relates to a DNA construct as well as host cells, expression systems, and transgenic plants which contain the heterologous DNA construct.

The DNA construct includes a DNA molecule encoding a hypersensitive response elicitor protein or polypeptide, a plant-expressible promoter operably coupled 5′ to the DNA molecule and which is effective to transcribe the DNA molecule in the tissues of cuttings, and a 3′ regulatory region operably coupled to the DNA molecule. Expression of the DNA molecule in such tissues imparts to a cutting resistance against desiccation.

Expression of such heterologous DNA molecules requires a suitable promoter which is operable in plant tissues. In some embodiments of the present invention, it may be desirable for the heterologous DNA molecule to be expressed in many, if not all, tissues. Such promoters yield constitutive expression of coding sequences under their regulatory control. Exemplary constitutive promoters include, without limitation, the nopaline synthase promoter (Fraley et al., Proc. Natl. Acad. Sci. USA 80:4803-4807 (1983), which is hereby incorporated by reference in its entirety) and the cauliflower mosaic virus 35S promoter (O'Dell et al., “Identification of DNA Sequences Required for Activity of the Cauliflower Mosaic Virus 35S Promoter,” Nature, 313(6005):810-812 (1985), which is hereby incorporated by reference in its entirety). Other constitutive plant promoters are continuously being identified and can be used in accordance with the present invention.

While constitutive expression is generally suitable for expression of the DNA molecule, it should be apparent to those of skill in the art that temporally or tissue regulated expression may also be desirable, in which case any regulated promoter can be selected to achieve the desired expression. Typically, the temporally or tissue regulated promoters will be used in connection with the DNA molecule that are expressed at only certain stages of development or only in certain tissues.

In another embodiment of the present invention, expression of the heterologous DNA molecule is directed in a tissue-specific manner or environmentally-regulated manner (i.e., inducible promoters). Tissue-specific promoters under developmental control include promoters that initiate transcription only in certain tissues.

Promoters useful for expression in leaf tissue include the Rubisco small subunit promoter.

Promoters useful for expression in flower tissues include the 5-enolpyruvylshikimate-3-phosphate synthase promoter (Benfy, et al., “Sequence Requirements of the 5-enolpyruvylshikimate-3-phosphate Synthase 5′-Upstream Region for Tissue-Specific Expression in Flowers and Seedlings,” The Plant Cell 2:849-856 (1990), which is hereby incorporated by reference in its entirety) and the tomato PG β-subunit promoter (U.S. Pat. No. 6,127,179 to DellaPenna et al., which is hereby incorporated by reference).

Examples of environmental conditions that may affect transcription by inducible promoters include anaerobic conditions, elevated temperature, or the presence of light. In some plants, it may also be desirable to use promoters which are responsive to pathogen infiltration or stress. For example, it may be desirable to limit expression of the protein or polypeptide in response to infection by a particular pathogen of the plant. One example of a pathogen-inducible promoter is the gst1 promoter from potato, which is described in U.S. Pat. Nos. 5,750,874 and 5,723,760 to Strittmayer et al., each of which is hereby incorporated by reference in its entirety.

Expression of the DNA molecule in isolated plant cells or tissue or whole plants also utilizes appropriate transcription termination and polyadenylation of mRNA. Any 3′ regulatory region suitable for use in plant cells or tissue can be operably linked to the first and second DNA molecules. A number of 3′ regulatory regions are known to be operable in plants. Exemplary 3′ regulatory regions include, without limitation, the nopaline synthase 3′ regulatory region (Fraley, et al., “Expression of Bacterial Genes in Plant Cells,” Proc. Nat'l. Acad. Sci. USA, 80:4803-4807 (1983), which is hereby incorporated by reference in its entirety) and the cauliflower mosaic virus 3′ regulatory region (Odell, et al., “Identification of DNA Sequences Required for Activity of the Cauliflower Mosaic Virus 35S Promoter,” Nature, 313(6005):810-812 (1985), which is hereby incorporated by reference in its entirety).

The promoter and a 3′ regulatory region can readily be ligated to the DNA molecule using well known molecular cloning techniques described in Sambrook et al., Molecular Cloning: A Laboratory Manual, Second Edition, Cold Spring Harbor Press, NY (1989), which is hereby incorporated by reference in its entirety.

One approach to transforming plant cells with a DNA molecule of the present invention is particle bombardment (also known as biolistic transformation) of the host cell. This can be accomplished in one of several ways. The first involves propelling inert or biologically active particles at cells. This technique is disclosed in U.S. Pat. Nos. 4,945,050, 5,036,006, and 5,100,792, all to Sanford, et al., each of which is hereby incorporated by reference in its entirety. Generally, this procedure involves propelling inert or biologically active particles at the cells under conditions effective to penetrate the outer surface of the cell and to be incorporated within the interior thereof. When inert particles are utilized, the vector can be introduced into the cell by coating the particles with the vector containing the heterologous DNA. Alternatively, the target cell can be surrounded by the vector so that the vector is carried into the cell by the wake of the particle. Biologically active particles (e.g., dried bacterial cells containing the vector and heterologous DNA) can also be propelled into plant cells. Other variations of particle bombardment, now known or hereafter developed, can also be used.

Another method of introducing the DNA molecule into plant cells is fusion of protoplasts with other entities, either minicells, cells, lysosomes, or other fusible lipid-surfaced bodies that contain the DNA molecule. Fraley, et al., Proc. Natl. Acad. Sci. USA, 79:1859-63 (1982), which is hereby incorporated by reference in its entirety.

The DNA molecule may also be introduced into the plant cells by electroporation. Fromm, et al., Proc. Natl. Acad. Sci. USA, 82:5824 (1985), which is hereby incorporated by reference in its entirety. In this technique, plant protoplasts are electroporated in the presence of plasmids containing the DNA molecule. Electrical impulses of high field strength reversibly permeabilize biomembranes allowing the introduction of the plasmids. Electroporated plant protoplasts reform the cell wall, divide, and regenerate.

Another method of introducing the DNA molecule into plant cells is to infect a plant cell with Agrobacterium tumefaciens or Agrobacterium rhizogenes previously transformed with the DNA molecule. Under appropriate conditions known in the art, the transformed plant cells are grown to form shoots or roots, and develop further into plants. Generally, this procedure involves inoculating the plant tissue with a suspension of bacteria and incubating the tissue for 48 to 72 hours on regeneration medium without antibiotics at 25-28° C.

Agrobacterium is a representative genus of the Gram-negative family Rhizobiaceae. Its species are responsible for crown gall (A. tumefaciens) and hairy root disease (A. rhizogenes). The plant cells in crown gall tumors and hairy roots are induced to produce amino acid derivatives known as opines, which are catabolized only by the bacteria. The bacterial genes responsible for expression of opines are a convenient source of control elements for chimeric expression cassettes. In addition, assaying for the presence of opines can be used to identify transformed tissue.

Heterologous genetic sequences such as a DNA molecule a hypersensitive response elicitor protein or polypeptide can be introduced into appropriate plant cells by means of the Ti plasmid of A. tumefaciens or the Ri plasmid of A. rhizogenes. The Ti or Ri plasmid is transmitted to plant cells on infection by Agrobacterium and is stably integrated into the plant genome. Schell, J., Science, 237:1176-83 (1987), which is hereby incorporated by reference in its entirety.

Plant tissue suitable for transformation include leaf tissue, root tissue, meristems, zygotic and somatic embryos, and anthers.

After transformation, the transformed plant cells can be selected and regenerated.

Preferably, transformed cells are first identified using, e.g., a selection marker simultaneously introduced into the host cells along with the DNA molecule of the present invention. Suitable selection markers include, without limitation, markers coding for antibiotic resistance, such as kanamycin resistance (Fraley, et al., Proc. Natl. Acad. Sci. USA, 80:4803-4807 (1983), which is hereby incorporated by reference in its entirety). A number of antibiotic-resistance markers are known in the art and other are continually being identified. Any known antibiotic-resistance marker can be used to transform and select transformed host cells in accordance with the present invention. Cells or tissues are grown on a selection media containing an antibiotic, whereby generally only those transformants expressing the antibiotic resistance marker continue to grow.

Once a recombinant plant cell or tissue has been obtained, it is possible to regenerate a full-grown plant therefrom. Thus, another aspect of the present invention relates to a transgenic ornamental plant that includes a heterologous DNA molecule encoding a hypersensitive response elicitor protein or polypeptide, wherein the heterologous DNA molecule is under control or a promoter that induces transcription of the DNA molecule in tissues of cuttings. Preferably, the DNA molecule is stably inserted into the genome of the transgenic plant of the present invention.

Plant regeneration from cultured protoplasts is described in Evans, et al., Handbook of Plant Cell Cultures, Vol. 1: (MacMillan Publishing Co., New York, 1983); and Vasil I. R. (ed.), Cell Culture and Somatic Cell Genetics is hereby incorporated by reference in its entirety.

It is known that practically all plants can be regenerated from cultured cells or tissues, including both monocots and dicots.

Means for regeneration vary from species to species of plants, but generally a suspension of transformed protoplasts or a petri plate containing transformed explants is first provided. Callus tissue is formed and shoots may be induced from callus and subsequently rooted. Alternatively, embryo formation can be induced in the callus tissue. These embryos germinate as natural embryos to form plants. The culture media will generally contain various amino acids and hormones, such as auxin and cytokinins. It is also advantageous to add glutamic acid and proline to the medium, especially for such species as corn and alfalfa. Efficient regeneration will depend on the medium, on the genotype, and on the history of the culture. If these three variables are controlled, then regeneration is usually reproducible and repeatable.

After the DNA molecule encoding the hypersensitive response elicitor protein or polypeptide is stably incorporated in transgenic plants, it can be transferred to other plants by sexual crossing or by preparing cultivars. With respect to sexual crossing, any of a number of standard breeding techniques can be used depending upon the species to be crossed. Cultivars can be propagated in accord with common agricultural procedures known to those in the field.

With respect to desiccation, complete protection against desiccation may not be conferred, but the severity of desiccation can be reduced. Desiccation protection inevitably will depend, at least to some extent, on other conditions such as storage temperatures, light exposure, etc. However, this method of controlling desiccation has the potential for eliminating some other treatments (i.e., additives to water, thermal regulation, etc.) which may contribute to reduced costs or, at least, substantially no increase in costs. Moreover, by controlling desiccation, it is also possible to enhance the longevity of flower blooms.

The methods of the present invention can be utilized to treat a wide variety of ornamental plants to control desiccation of cuttings removed therefrom as well as enhance the longevity of flowers. Ornamental plants can be either monocots or dicots. Cuttings include stems, leaves, flowers, or combinations thereof.

In addition to treatment with hypersensitive response elicitor proteins or polypeptides, as well as transgenic expression thereof in tissues of cuttings, cuttings or ornamental plants (transgenic or otherwise) can also be treated with ethylene action inhibitors of the types disclosed in U.S. Pat. No. 6,194,350 to Sisler, U.S. Pat. No. 6,153,559 to Heiman, and U.S. Pat. No. 5,518,988 to Sisler et al., each of which is hereby incorporated by reference in its entirety. Such treatment can occur before harvest, after harvest, or both. One commercially available ethylene-action inhibitor is EthylBloc® (1-methylcyclopropene, available from AgroFresh Inc. and Floralife Inc.).

### EXAMPLES

The following examples are intended to illustrate, but by no means are intended to limit, the scope of the present invention as set forth in the appended claims.

### Example 1—Increased Flower Quality and Longevity of Roses from Postharvest Application of EBC-151 (Messenger®)

Mature rose plants were treated with Messenger® (coded as EBC-151) by foliar sprays and postharvest treatment to improve flower quality and longevity. The trial was established in a commercial rose greenhouse in Villa Guerrero, Mexico. The rose variety in this trial was Vega. Individual plot beds contained approximately 44 mature plants arranged in two rows; each plot was replicated 4 times and measured 80 cm wide by 15.4 m long. EBC-151 treatments were applied with a CO₂-powered backpack sprayer calibrated to deliver 430 l/Ha at 90 psi. Treatment rates and timings in this trial are shown in Table 1 below.

TABLE 1
Application rates and treatment schedule for EBC-151 to Vega roses

| | EBC-151 | |
|---|---|---|
| Treatment | Application Rate | Treatment Details |
| 1 | 250 g/Ha | 8 applications at approximately |
| | | 14-d intervals |
| 2 | 250 g/Ha + 3.33 g/L | 8 applications at approximately |
| | postharvest spray | 14-d intervals followed by a |
| | | postharvest spray to 10 |
| | | commercially-harvested |
| | | flower/stems within |
| | | 1 hour of cutting |
| 3 | 150 g Ha + 350 g/Ha | 150 g/Ha applied 5 times followed |
| | | by 350 g/Ha applied 3 times |
| | | at the same 14-d schedule, |
| | | no postharvest application |
| 4 | 150 g/Ha + 350 g/Ha + | 150 g/Ha applied 5 times followed |
| | 3.33 g/L | by 350 g/Ha applied 3 times |
| | postharvest spray | at the same 14-d schedule followed |
| | | by a postharvest spray to |
| | | 10 commercially-harvested |
| | | flower/stems within 1 hour of |
| | | cutting |
| 5 | 3.33 g/L | Postharvest spray only to 10 |
| | postharvest spray only | commercially-harvested |
| | | flower/stems within 1 hour of |
| | | cutting |
| 6 | N/a | Untreated with EBC-151 |

Preharvest applications of each EBC-151 treatment were repeated at approximately 14-d intervals. After the fifth preharvest application, 10 mature flower/stems were randomly selected from each treatment and evaluated. Treatment effects were evaluated on cut flowers by assessing the number of open flowers and the number of “straight” stems on each flower/stem. An “open” flower was determined to conform to commercial standards for sale by having flower petals extended. Flower petals judged as partially extended were rated as “not open”. Straight stems were evaluated as conforming to commercial standard of acceptability for sale. Results for this evaluation are shown in Table 2 below. No postharvest applications of EBC-151 were made to flower/stems harvested after the fifth application of EBC-151.

TABLE 2
Response of cut Vega roses to treatment with
EBC-151 (five applications only)

| | Number | | | Number of |
|---|---|---|---|---|
| Treat- | of | Number of | Percent “open” | Flowers with |
| ment | Flowers | “Open” Flowers | Flowers | “Straight” Stems |
| 1 | 10 | 10 | 100 | 10 |
| 3 | 10 | 2 | 20 | 6 |
| 6 | 10 | 1 | 10 | 4 |

Additional preharvest treatments continued with three more applications (for a total of eight applications). Following the eighth application, an additional 10 mature flower/stems were then randomly selected from each treatment and evaluated in the same manner as had been done after the fifth application. Immediately after cutting (within 1 hour) a single postharvest treatment of EBC-151 was applied at the rate of 3.33 g/L (100 ppm a.i.) to the cut flower/stems harvest from Treatments 2, 4 and 5. The postharvest spray was applied by completely misting each flower/stem with the EBC-151 solution. Sixteen days after postharvest treatment, the number of open flowers and number of flowers with “straight” stems were determined for each treatment. Results for this evaluation are shown in Table 3 below.

TABLE 3
Response of cut Vega roses to treatment with EBC-151
(eight preharvest and one postharvest application)

| | Number | | | Number of |
|---|---|---|---|---|
| Treat- | of | Number of | Percent “open” | Flowers with |
| ment | Flowers | “Open” Flowers | Flowers | “Straight” Stems |
| 1 | 10 | 9 | 90 | 8 |
| 2 | 10 | 10 | 100 | 8 |
| 3 | 10 | 9 | 90 | 9 |
| 4 | 10 | 10 | 100 | 9 |
| 5 | 10 | 3 | 30 | 1 |
| 6 | 10 | 2 | 20 | 2 |

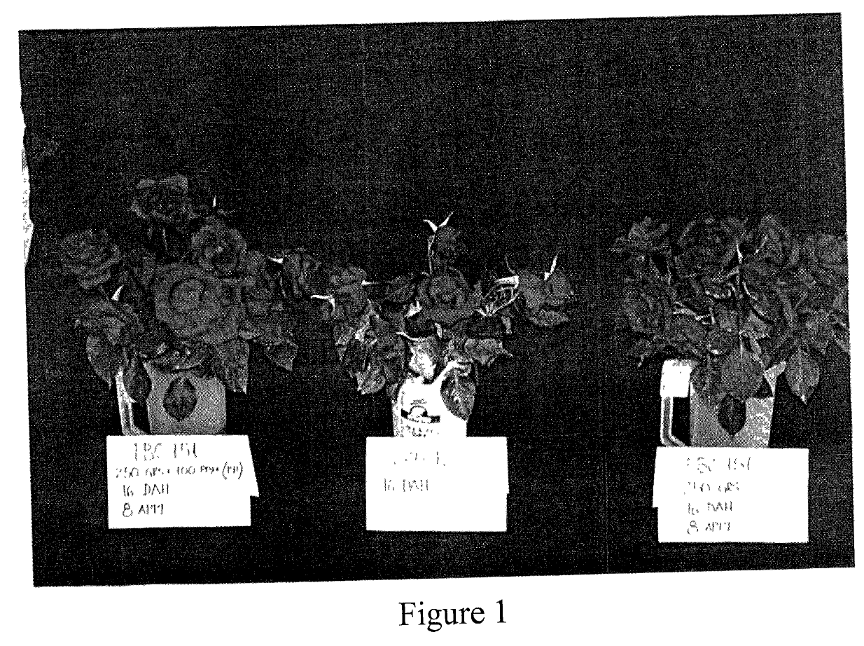

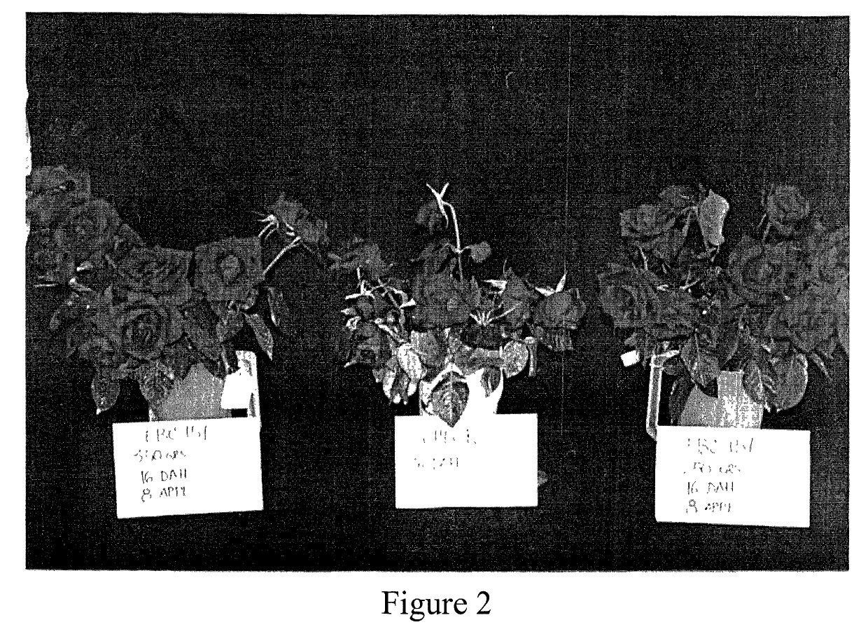

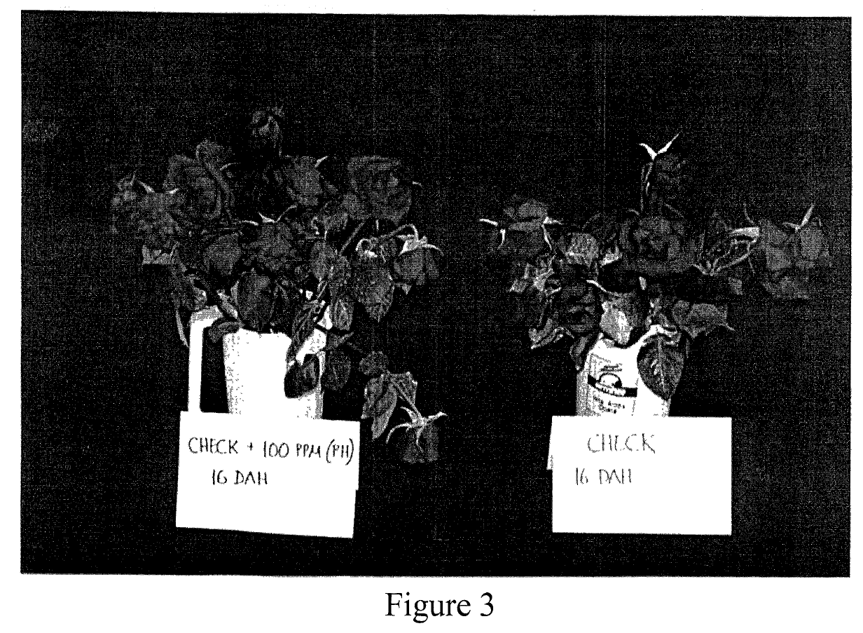

Visual observations of cut roses 16 days after postharvest treatment were made for treatments that received postharvest applications of EBC-151. Roses that had been treated with the postharvest application of EBC-151 appeared to have substantially greater longevity than those that had not received the postharvest treatment (FIGS. 1-3).

Results of this trial demonstrated a treatment effect for application of EBC-151 (Messenger®) to roses. The effect was seen in a substantially greater increase in the number of open flowers at harvest. This effect is of significant commercial benefit to rose growers. In addition, the postharvest application of EBC-151 to cut roses resulted in substantially extending the “shelf life” of the cut roses.

Although the invention has been described in detail for the purpose of illustration, it is understood that such detail is solely for that purpose, and variations can be made therein by those skilled in the art without departing from the spirit and scope of the invention which is defined by the following claims.

What is claimed:

1. A method of inhibiting desiccation of cuttings from ornamental plants comprising:
treating an ornamental plant with a hypersensitive response elicitor protein or polypeptide under conditions effective to inhibit desiccation of a cutting from the ornamental plant after the cutting is removed from the ornamental plant.

2. The method of claim 1, wherein said treating comprises topically applying the hypersensitive response elicitor protein or polypeptide to the ornamental plant.

3. The method of claim 1, wherein the hypersensitive response elicitor protein or polypeptide is derived from a plant pathogen.

4. The method of claim 3, wherein the plant pathogen is selected from the group consisting of Erwinia, Pseudomonas, Ralstonia, Xanthomonas, Clavibacter, and Phytophthora.

5. The method of claim 1, wherein the ornamental plant is a monocot or a dicot.

6. The method of claim 1 further comprising:
removing a cutting from the treated ornamental plant and
applying a hypersensitive response elicitor to the removed cutting.

7. The method of claim 1, wherein the cutting comprises a stem, a leaf, a flower, or combinations thereof.

8. A cutting which has been removed from an ornamental plant treated with a hypersensitive response elicitor protein or polypeptide, wherein the cutting is characterized by greater resistance to desiccation as compared to a cutting removed from an untreated ornamental plant.

9. The cutting according to claim 8, wherein the cutting comprises a stem, a leaf, a flower, or combinations thereof.

10. The cutting of claim 8, wherein the hypersensitive response elicitor protein or polypeptide is derived from a plant pathogen.

11. The cutting of claim 10, wherein the plant pathogen is selected from the group consisting of Erwinia, Pseudomonas, Ralstonia, Xanthomonas, Clavibacter, and Phytophthora.

12. The cutting of claim 8, wherein the ornamental plant is a monocot or a dicot.

13. A method of promoting early flowering of an ornamental plant comprising:
treating an ornamental plant with a hypersensitive response elicitor protein or polypeptide under conditions effective to promote early flowering of the ornamental plant.

14. The method of claim 13, wherein said treating comprises topically applying the hypersensitive response elicitor to the ornamental plant.

15. The method of claim 13, wherein the hypersensitive response elicitor protein or polypeptide is derived from a plant pathogen.

16. The method of claim 15, wherein the plant pathogen is selected from the group consisting of Erwinia, Pseudomonas, Ralstonia, Xanthomonas, Clavibacter, and Phytophthora.

17. The method of claim 13, wherein the ornamental plant is a monocot or a dicot.

18. A method of harvesting a cutting from an ornamental plant comprising:
treating an ornamental plant with a hypersensitive response elicitor protein or polypeptide and
harvesting a cutting from the treated ornamental plant.

19. The method of claim 18, wherein said treating comprises topically applying the hypersensitive response elicitor protein or polypeptide to the ornamental plant.

20. The method of claim 18, wherein the hypersensitive response elicitor protein or polypeptide is derived from a plant pathogen.

21. The method of claim 20, wherein the plant pathogen is selected from the group consisting of Erwinia, Pseudomonas, Ralstonia, Xanthomonas, Clavibacter, and Phytophthora.

22. The method of claim 18, wherein the ornamental plant is a monocot or a dicot.

23. The method of claim 18 further comprising:
applying a hypersensitive response elicitor protein or polypeptide to the harvested cutting.

24. The method of claim 18, wherein the cutting comprises a stem, a leaf, a flower, or combinations thereof.

25. A method of harvesting a cutting from an ornamental plant comprising:
harvesting a cutting from an ornamental plant and
treating the harvested cutting with a hypersensitive response elicitor protein or polypeptide.

26. The method of claim 25, wherein said treating comprises topically applying the hypersensitive response elicitor protein or polypeptide to the cutting.

27. The method of claim 25, wherein the hypersensitive response elicitor protein or polypeptide is derived from a plant pathogen.

28. The method of claim 27, wherein the plant pathogen is selected from the group consisting of Erwinia, Pseudomonas, Ralstonia, Xanthomonas, Clavibacter, and Phytophthora.

29. The method of claim 25, wherein the ornamental plant is a monocot or a dicot.

30. The method of claim 25, wherein the cutting comprises a stem, a leaf, a flower, or combinations thereof.

31. A method of inhibiting desiccation of cuttings from ornamental plants comprising:
removing a cutting from an ornamental plant and
treating the removed cutting with a hypersensitive response elicitor protein or polypeptide under conditions effective to inhibit desiccation of the removed cutting.

32. The method of claim 31, wherein said treating comprises topically applying the hypersensitive response elicitor protein or polypeptide to the cutting.

33. The method of claim 31, wherein the hypersensitive response elicitor protein or polypeptide is derived from a plant pathogen.

34. The method of claim 33, wherein the plant pathogen is selected from the group consisting of Erwinia, Pseudomonas, Ralstonia, Xanthomonas, Clavibacter, and Phytophthora.

35. The method of claim 31, wherein the ornamental plant is a monocot or a dicot.

36. The method of claim 31, wherein the cutting comprises a stem, a leaf, a flower, or combinations thereof.

37. A cutting which has been removed from an ornamental plant, wherein the cutting has been treated with a hypersensitive response elicitor protein or polypeptide and wherein the cutting is characterized by greater resistance to desiccation as compared to an untreated cutting removed from the ornamental plant.

38. The cutting according to claim 37, wherein the cutting comprises a stem, a leaf, a flower, or combinations thereof.

39. The cutting of claim 37, wherein the hypersensitive response elicitor protein or polypeptide is derived from a plant pathogen.

40. The cutting of claim 39, wherein the plant pathogen is selected from the group consisting of Erwinia, Pseudomonas, Ralstonia, Xanthomonas, Clavibacter, and Phytophthora.

41. The cutting of claim 37, wherein the ornamental plant is a monocot or a dicot.

42. A method of inhibiting desiccation of cuttings from ornamental plants comprising:
providing a transgenic ornamental plant or plant seed transformed with a DNA molecule encoding a hypersensitive response elicitor polypeptide or protein and
growing the transgenic ornamental plant or transgenic ornamental plant produced from the transgenic ornamental plant seed under conditions effective to inhibit desiccation in a cutting removed from the transgenic plant.

43. The method of claim 42, wherein the hypersensitive response elicitor protein or polypeptide is derived from a plant pathogen.

44. The method of claim 43, wherein the plant pathogen is selected from the group consisting of Erwinia, Pseudomonas, Ralstonia, Xanthomonas, Clavibacter, and Phytophthora.

45. The method of claim 42, wherein the transgenic ornamental plant is a monocot or a dicot.

46. The method of claim 42, wherein the cutting is a stem, a leaf, a flower, or combinations thereof.

47. The method of claim 42 further comprising:
removing a cutting from the transgenic ornamental plant and
applying a hypersensitive response elicitor protein or polypeptide to the removed cutting.

48. The method of claim 42, wherein the hypersensitive response elicitor protein or polypeptide is expressed in tissues of the cutting.

49. A method of promoting early flowering of an ornamental plant comprising:
providing a transgenic ornamental plant or plant seed transformed with a DNA molecule encoding a hypersensitive response elicitor polypeptide or protein and
growing the transgenic ornamental plant or transgenic ornamental plant produced from the transgenic ornamental plant seed under conditions effective to promote early flowering of the transgenic ornamental plant.

50. The method of claim 49, wherein the hypersensitive response elicitor protein or polypeptide is derived from a plant pathogen.

51. The method of claim 50, wherein the plant pathogen is selected from the group consisting of Erwinia, Pseudomonas, Ralstonia, Xanthomonas, Clavibacter, and Phytophthora.

52. The method of claim 49, wherein the transgenic ornamental plant is a monocot or a dicot.

53. The method of claim 49, wherein the cutting is a stem, a leaf, a flower, or combinations thereof.

54. The method of claim 49, wherein the hypersensitive response elicitor protein or polypeptide is expressed in flower tissues.

55. A method of harvesting a cutting from an ornamental plant comprising:
providing a transgenic ornamental plant or plant seed transformed with a DNA molecule encoding a hypersensitive response elicitor polypeptide or protein;
growing the transgenic ornamental plant or transgenic ornamental plant produced from the transgenic ornamental plant seed under conditions; and
harvesting a cutting from the grown transgenic ornamental plant, wherein the cutting exhibits a reduced susceptibility to desiccation as compared to cuttings removed from non-transgenic ornamental plants.

56. The method of claim 55, wherein the hypersensitive response elicitor protein or polypeptide is derived from a plant pathogen.

57. The method of claim 56, wherein the plant pathogen is selected from the group consisting of Erwinia, Pseudomonas, Ralstonia, Xanthomonas, Clavibacter, and Phytophthora.

58. The method of claim 55, wherein the transgenic ornamental plant is a monocot or a dicot.

59. The method of claim 55, wherein the cutting is a stem, a leaf, a flower, or combinations thereof.

60. The method of claim 55 further comprising:
applying a hypersensitive response elicitor protein or polypeptide to the harvested cutting.

61. The method of claim 55, wherein the hypersensitive response elicitor protein or polypeptide is expressed in tissues of the cutting.

62. A cutting which has been removed from a transgenic ornamental plant which expresses a heterologous hypersensitive response elicitor protein or polypeptide, wherein the cutting is characterized by greater resistance to desiccation as compared to a cutting removed from a non-transgenic ornamental plant.

63. The cutting of claim 62, wherein the cutting comprises a stem, a leaf, a flower, or combinations thereof.

64. The cutting of claim 62, wherein the hypersensitive response elicitor protein or polypeptide is derived from a plant pathogen.

65. The cutting of claim 64, wherein the plant pathogen is selected from the group consisting of Erwinia, Pseudomonas, Ralstonia, Xanthomonas, Clavibacter, and Phytophthora.

66. The cutting of claim 62, wherein the transgenic ornamental plant is a monocot or a dicot.

67. The cutting of claim 62, wherein the hypersensitive response elicitor protein or polypeptide is expressed in tissues of the cutting.

68. A method of enhancing the longevity of flower blooms on ornamental plant cuttings, the method comprising:
providing a transgenic ornamental plant or plant seed transformed with a DNA molecule encoding a hypersensitive response elicitor polypeptide or protein and
growing the transgenic ornamental plant or transgenic ornamental plant produced from the transgenic ornamental plant seed under conditions effective to enhancing the longevity of flower blooms on cuttings removed therefrom.

69. The method of claim 68, wherein the hypersensitive response elicitor protein or polypeptide is derived from a plant pathogen.

70. The method of claim 69, wherein the plant pathogen is selected from the group consisting of Erwinia, Pseudomonas, Ralstonia, Xanthomonas, Clavibacter, and Phytophthora.

71. The method of claim 68, wherein the transgenic ornamental plant is a monocot or a dicot.

72. The method of claim 68, wherein the cutting is a stem, a leaf, a flower, or combinations thereof.

73. The method of claim 68, wherein the hypersensitive response elicitor protein or polypeptide is expressed in flower tissues.

74. The method of claim 68 further comprising:
harvesting a cutting from the transgenic ornamental plant and
applying a hypersensitive response elicitor protein or polypeptide to the harvested cutting.

75. A method of enhancing the longevity of flower blooms on ornamental plant cuttings, the method comprising:
treating an ornamental plant with a hypersensitive response elicitor protein or polypeptide under conditions effective to enhancing the longevity of flower blooms on cuttings removed therefrom.

76. The method of claim 75, wherein said treating comprises topically applying the hypersensitive response elicitor to the ornamental plant.

77. The method of claim 75, wherein the hypersensitive response elicitor protein or polypeptide is derived from a plant pathogen.

78. The method of claim 77, wherein the plant pathogen is selected from the group consisting of Erwinia, Pseudomonas, Ralstonia, Xanthomonas, Clavibacter, and Phytophthora.

79. The method of claim 75, wherein the ornamental plant is a monocot or a dicot.

80. The method of claim 75 further comprising:
harvesting a cutting from the treated ornamental plant and
applying a hypersensitive response elicitor protein or polypeptide to the harvested cutting.

81. A method of enhancing the longevity of flower blooms on ornamental plant cuttings, the method comprising:
harvesting a cutting from an ornamental plant and
treating the harvested cutting with a hypersensitive response elicitor protein or polypeptide under conditions effective to enhancing the longevity of flower blooms on the harvested cutting.

82. The method of claim 81, wherein said treating comprises topically applying the hypersensitive response elicitor to the ornamental plant.

83. The method of claim 81, wherein the hypersensitive response elicitor protein or polypeptide is derived from a plant pathogen.

84. The method of claim 83, wherein the plant pathogen is selected from the group consisting of Erwinia, Pseudomonas, Ralstonia, Xanthomonas, Clavibacter, and Phytophthora.

85. The method of claim 81, wherein the ornamental plant is a monocot or a dicot.
